(19) United States
(12) Patent Application Publication
(10) Pub. No.: US 2023/0310001 A1
(43) Pub. Date: Oct. 5, 2023

(54) ANASTOMOTIC COUPLING DEVICE

(71) Applicant: THE JOHNS HOPKINS UNIVERSITY, Baltimore, MD (US)

(72) Inventors: Justin Sacks, Baltimore, MD (US); Helen Xun, Baltimore, MD (US); Caitlin Hicks, Baltimore, MD (US); Sung Hoon Kang, Baltimore, MD (US); Gerald Brandacher, Baltimore, MD (US); Christopher Shallal, Charlotte, NC (US)

(21) Appl. No.: 18/013,875

(22) PCT Filed: Jul. 7, 2021
(86) PCT No.: PCT/US2021/040749

§ 371 Date: Dec. 29, 2022

Related U.S. Application Data
(60) Provisional application No. 63/048,897, filed on Jul. 7, 2020.

(51) Int. Cl. A61B 17/11

(52) U.S. Cl. CPC A61B 17/1114; A61B 2017/00982; A61B 2017/1107; A61B 2017/1132

(57) ABSTRACT

A coupler is provided that includes a member such as a tube having a passageway and at least one end of the tube is inserted into a vessel. Upon insertion, the tube engages with inner walls of the vessel to inhibit movement of the coupler and/or adjoin tissue or a vessel.

This application claims the benefit of priority under 35 U.S.C. §119(e) to U.S. Provisional Application No. 63/048,897 filed Jul. 7, 2020, the entire contents of which is incorporated herein by reference in its entirety.

### 1. TECHNICAL FIELD

The present disclosure relates to an anastomotic coupling device, and more particularly, to an anastomotic coupling device having protrusions extending from a peripheral surface of the device.

### 2. BACKGROUND

Anastomosis is used across a variety of surgical disciplines to provide a surgical connection between adjacent tubular structures. In many medical procedures, a vessel, duct, or other tubular structure must be joined with another vessel, duct, or other tubular structure to establish a connection therebetween. This procedure typically requires multiple tools and also requires a high skill level of a surgeon. Conventionally, this anastomosis surgery consists of manually suturing two tubular structures together around an opening therebetween. This manual process is time-consuming and requires a high skill level of a surgeon which causes varying results of the procedure. Additionally, the manual suturing and connection of the tubular structures requires a substantial healing time for the tubular structures.

One developed technique in this field is a mechanical coupling that creates a compressive anastomosis between tissues. The coupling is placed over the tissues to be joined and then over time, new tissue is formed therebetween. However, such a technique requires accurate placement of the couplings and otherwise leads to leakage. In addition, the joint strength of the coupling is limited until the tissues are healed. Another developed technique uses adhesives instead of requiring manual suturing. However, adhesives do not provide sufficient joint strength in comparison to a mechanical coupling.

FIG. 1 shows another developed coupler device of the prior art. In particular, FIG. 1 illustrates a mechanical coupling 100 that joins the inner walls of each tubular structure 105. However, as shown, this device still requires the manual suturing 110 to secure the joint connection. Although these described techniques may be used for various surgical procedures, they are also contemplated for use in microvascular surgical application. However, in performing a microvascular anastomotic procedure, the challenge and skill level further increases and the process also requires the use of a microscope, such as a surgical microscope. The manual suturing process also becomes more time-consuming and requires a high accuracy for consistent results. Accordingly, there is a need for a more reliable alternative to suturing techniques and an alternative that is easier to implement with consistent results.

### SUMMARY

In one aspect, we now provide an anastomotic device. In preferred systems, an anastomotic device can reliably secure or couple a first vessel to a second vessel without need for use of any sutures. In a particularly preferred system, the coupler also can prevent or at least inhibit undesired rotational or lateral movement of the joined vessels.

In related aspects, a device is provided that includes a connector and an incision seal system that may be configured and used to join, seal, or otherwise couple a first vessel to a second vessel without the use of sutures.

Preferred sutureless devices also minimize manipulation, positioning, or preparation of vessels prior to anastomosis, which could result in compromise of vessel tissue or anastomotic integrity.

A preferred sutureless coupler may include a tube member having a passageway formed therethrough. In use, at least one end of the tube is inserted into a vessel where the tube can engage the vessel’s inner walls to inhibit or prevent undesired movement of the coupler. In one exemplary embodiment, the tube includes a plurality of protrusions or anchors such as may be formed for example integral to the tube including but not limited to a peripheral surface such as along at least one or each end of the tube. In certain configurations, the protrusions or anchors of each tube end may have opposed or mixed orientations to facilitate grasping of two vessels being joined by the device.

In another exemplary embodiment, the tube may be formed of an expandable material. In such an exemplary embodiment, the tube may expand upon a particular increase in manual force, temperature, pressure, flow detection, magnetic field, light, sound wave and/or pH after insertion into the vessel. The engagement of the tube with the inner walls of the vessel can inhibit or prevent lateral and rotational movement of the sutureless coupler.

In a further aspect, an anastomotic device is provided and a method for operating the same in which vessels are joined and held in place by a tube having protrusions extending from a peripheral surface thereof thus providing a more stable joint connection using a simplified device while also maintaining a tubular flow through the vessels without leakage.

According to another aspect of the present disclosure, an anastomotic device may include a tube having a passageway formed therethrough and a plurality of protrusions extending from the tube such as for example a peripheral surface of one or each end of the tube. At least one end of the tube is inserted into a vessel and the plurality of protrusions extending therefrom engage with inner walls of the vessel locking the anastomotic device in the vessel.

In an exemplary embodiment, the plurality of protrusions at a first end of the tube extend in an opposing direction to the plurality of protrusions at a second end of the tube. An insertion direction of the at least one end of the tube is in a protruding direction of the plurality of protrusions. Additionally, each end of the tube may include 1 to 20 or more rows of protrusions on the peripherals surface thereof. The protrusions may extend at an angle ranging from 1 degree to 90 degrees from the peripheral surface of the tube.

The protrusions may be substantially uniform in one or more respect, and/or may vary in one or more respects.

For instance, the protrusions of a device each may be of substantially the same size and configuration, or a device may contain protrusions that differ in size and/or configuration. For example, a device may comprise protrusions that differ in height (distance extending from device planar surface), and/or length and/or shape. A device also may contain protrusions that are positioned substantially in a unidirectional (parallel) to the anastomosis, or a device may contain protrusions that have a dispersion of diverging orientations, i.e. bidirectional or multi-directional with respect to the anastomosis.

Further, the second end of the tube may have a diameter greater than the diameter of the first end of the tube. A tube also suitably may be tapered in outside diameter from ends to a center section while maintaining a substantially fixed bore diameter throughout. A diameter of the tube may gradually increase from the first end thereof to the second end thereof. Alternatively, the ends of the tube may be branched into multiple outlets. The tube may also be formed of a biocompatible and/or biodegradeable and/or bioerodable materials. An inner diameter (includes largest cross-sectional dimension in non-circular cross-section devices) of the tube may be in ‘a range of about 0.1 mm to 20 mm, or in certain embodiments an inner diameter of 0.1 to 6, 7, 8, 9 10 cm. In particular, for use of a device with larger vessels such as a subject’s colon, a device having an inner diameter of 1 or more cm (such as up to 1, 2, 3, 4, 5, 6, 7, 8, 9, 10, 11 or 12 cm or more) may be useful. A distance from each end of the tube to the respective protrusions may be 0 to a mid-length of the length of the tube. It is understood that references herein to diameter of a device herein include largest cross-sectional dimension in devices that have non-circular cross-sections.

According to another aspect of the present disclosure, an anastomotic system is provided. The system may include a tube having a passageway formed thereof, a plurality of protrusions extending from a peripheral surface of each end of the tube, and a manipulator that can facilitate engagement and placement of the device. In one embodiment, a manipulator may have a clamp at a distal end thereof to grasp the tube. In another aspect, a manipulator does not utilize a clamp. A first end and a second end of the tube are inserted by the manipulator into a first vessel and a second vessel, respectively, and the protrusions of each end of the tube engage with inner walls of a respective vessel to join the first and second vessels.

In an exemplary embodiment, the plurality of protrusion at the first end of the tube extend in an opposing direction to the plurality of protrusions at the second end of the tube. Additionally, the manipulator may include a cover attached to the clamp to cover the protrusions during insertion into the first and second vessels and allows for manipulation of the tube within the vessels without damaging the structure or surface of the vessels. The cover may be removed when the clamp is released after insertion of the first and second ends of the tube into the respective vessel, thus pulling the first and second vessels together. An insertion direction of each of the first and second ends of the tube may be in a protruding direction of respective protrusions.

According to yet another aspect of the present disclosure, an anastomotic coupling method is provided. The method may include grasping, by a clamp, a tube having a passageway formed therethrough and a plurality of protrusions extending from a peripheral surface of each end of the tube. A first end of the tube may then be inserted into a first vessel to engage the protrusions formed thereon with inner walls of the first vessel. A second end of the tube may be inserted into a second vessel to engage the protrusions formed thereon with inner walls of the second vessel. The tube may then be released from the clamp to join the first and second vessels.

According to an exemplary embodiment, the grasping of the tube may further include attaching a cover to the clamp to cover the protrusions during insertion into the vessels. The release of the tube may include releasing the clamp and removing the cover together with the clamp to expose the tube into the vessels thus causing the protrusions to engage with the inner walls of the vessels. Additionally, the removing of the cover causes a central force which pulls the first and second vessels together without requiring the additional process of suturing.

In other aspects, methods are provided for joining one or more vessels (particularly two vessels) of a subject using a device as disclosed herein. The present device may be utilized in a wide range of surgical procedures and to join or otherwise attach or contact with a range of tissue including arteries; veins; blood vessels; lymphatics; any duct including pancreatic ducts, cystic ducts, hepatic duct, bile duct, ureters, vas deferens; fallopian tubes; and bowels, including small intestine, large intestine including colon.

As the term “vessel” is used herein, unless otherwise specified, the term embraces any of such tissue (i.e. including without limitation arteries; veins; blood vessels; lymphatics; any duct including pancreatic ducts, cystic ducts, hepatic duct, bile duct, ureters, vas deferens; fallopian tubes; and bowels, including small intestine, large intestine including colon) and embraces tubular structures.

The terms “protrusion” and “anchor” of a coupling device are used interchangeably herein and designate the same (i.e. 215 in the figures).

The anastomotic device described herein is useful across various surgical disciplines including, but not limited to, vascular surgery, plastic and reconstructive surgery, oral and maxillofacial surgery, neurosurgery, ophthalmology, urology, bowel surgery, interventional radiology, and the like. The device in various configurations including varying cross-sectional dimensions may be used in surgical applications of a micro size up to a macro size. The device is also not limited to connecting tubular structures within the body. The device may also be used to connect an interior vessel to an exterior vessel. For example, the device may connect an interior tubular structure with an exterior colonoscopy bag or other type of exterior tubular structure.

Notably, the present invention is not limited to the combination of the device elements as listed above and may be assembled in any combination of the elements as described herein.

As referred to herein, the term “sutureless” means that the device can be used to join a tubular structure without the need to use of sutures, stitches, staples, or other manual, seam-forming connectors in order to secure attachment of the device to the vessel. A device can be empirically assessed to be “sutureless” herein if the device passes the gravity test as described below and depicted in FIG. 7A. Particularly preferred sutureless devices also pass the engagement test described below and depicted in FIG. 7B or still more preferably also pass the lock test described below and depicted in FIG. 7C. It also will be understood that an optional one or more stitches (e.g. a safety stitch) may be used with a device and the device still would be considered “sutureless” herein provided the device can join a vessel without the need of a stitch, as might be empirically assessed by the gravity test of FIG. 7A and as specified below, or further assessed by the engagement test or lock test of FIGS. 7B and 7C respectively and as specified below.

As referred to herein, the “gravity test” (the defined term can be indicated herein by capitalization i.e. Gravity Test) is the test shown in FIG. 7A and described below where a device is considered to pass the Gravity Test where the device remains connected to a vessel against gravity with one end of the vessel being suspended vertically (e.g., via tweezers) and the device remains engaged in the bottom end of the vessel. A device would be considered to fail the Gravity Test where upon such vertical suspension the device does not remain engaged in the bottom end of the vessel.

As referred to herein the “engagement test” (the defined term can be indicated herein by capitalization i.e. Engagement Test) is the test shown in FIG. 7B and described below where a device is considered to pass the Engagement Test where the device remains in place (connected to the vessel) while the vessel is maneuvered (such as via tweezers as shown in FIG. 7) without stretching the vessel beyond static length. A device would be considered to fail the Engagement Test where upon such manipulation (e.g. by tweezers as shown in FIG. 7B) the device does not remain engaged in the vessel.

As referred to herein, the “lock test” (the defined term can be indicated herein by capitalization i.e. Lock Test) is the test shown in FIG. 7C and described below where a device is considered to pass the Lock Test where the device is and remains engaged with a vessel while the engaged device and vessel are pulled away from each other beyond the vessel’s static length by an additional 10 percent of force beyond the force of the Engagement Test, i.e. an additional 10 percent of force beyond the force required to extend the vessel but without stretching the vessel beyond static length. A device would be considered to fail the Lock Test where upon application of such a 10%-additional force (e.g. such force exerted by tweezers as shown in FIG. 7C) the device does not remain engaged in the vessel.

Generally preferred devices pass the Gravity Test. Particularly preferred devices pass both the Gravity Test and Engagement Test. In certain aspects, particularly preferred devices pass the Lock Test.

In certain preferred aspects, a device will have substantially the same diameter for substantially the full length of the device, for example, a device will have an inner diameter that does not vary by more than 10, 8, 6, 5, 4, 3, 2 or 1 percent over at least about 50, 60, 70, 80, 90 95, 98 or the entire length of the device. In certain related aspects, a device will not contain a protruding (greater cross-section dimension) area or ridge along it middle portion, or elsewhere along the device length.

In certain aspects, a device will not include a shape memory material or alloy such as nitinol or NiTi, or shape memory polymer or gel.

In other certain aspects, a device will be formed at least in part from a shape memory material or alloy such as nitinol or NiTi, or shape memory polymer or gel, for example in such aspects at least or up to about 5, 10, 20, 30, 40, 50, 60, 70, 80, 90, 95, 98, 99 weight percent or more (including 100 weight percent) of the material forming a present device is a shape memory material or alloy such as nitinol or NiTi, or shape memory polymer or gel.

In certain aspects, a device will not include poly (lactic-co-glycolic acid) (PLGA) or polycaprolactone (PCL) or other material that may degrade substantially (at least 25, 40, 50, 60 or 70 weight percent over time period such as 1, 2, or 3 days or 1, 2, 3, 4, 5, 6, 7 or 8 weeks).

In other certain aspects, a device will include poly (lactic-co-glycolic acid) (PLGA) or polycaprolactone (PCL) or other material that may degrade substantially (at least 25, 40, 50, 60 or 70 weight percent over time period such as 1, 2, or 3 days or 1, 2, 3, 4, 5, 6, 7 or 8 weeks), for example in such aspects at least or up to about 5, 10, 20, 30, 40, 50, 60, 70, 80, 90, 95, 98, 99 weight percent or more (including 100 weight percent) of the material forming a present device is poly (lactic-co-glycolic acid) (PLGA) or polycaprolactone (PCL) or other material that may degrade substantially (at least 25, 40, 50, 60 or 70 weight percent over time period such as 1, 2, or 3 days or 1, 2, 3, 4, 5, 6, 7 or 8 weeks).

In certain preferred aspects, a coupler or device as disclosed herein may comprise as a material of construction one or more of a biocompatible polymeric, copolymeric, metallic, or composite material doped or undoped for radio-opacity. In particular, preferred coupler and devices as disclosed herein may comprise as a material of construction one or more of PEEK, polyurethane, polycarbonate, PTFE, acrylates, or derivatives thereof which may be coated or surface derivatized including for enhanced biocompatibility suitable for in vivo fluid and tissue exposure.

Other aspects of the invention are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

The embodiments herein may be better understood by referring to the following description in conjunction with the accompanying drawings in which like reference numerals indicate identically or functionally similar elements, of which:

FIG. 1 illustrates a flow coupler device according to the prior art;

FIG. 2A illustrates an anastomotic device according to an exemplary embodiment of the present disclosure;

FIG. 2B illustrates the anastomotic device inserted into a vessel according to an exemplary embodiment of the present disclosure;

FIG. 2C illustrates the sizing relationship of the vessel and anastomotic device according to an exemplary embodiment of the present disclosure;

FIGS. 3A-3B illustrates the protrusions extending from a peripheral surface of the anastomotic device according to an exemplary embodiment of the present disclosure;

FIGS. 4A-4D illustrate the tubular structure of the anastomotic device according to an exemplary embodiment of the present disclosure;

FIG. 5 illustrates an anastomotic device having a multiple outlets according to an exemplary embodiment of the present disclosure;

FIGS. 6A-6C illustrate the manipulator of an anastomotic system according to an exemplary embodiment of the present disclosure; and

FIGS. 7A-7C provide a showing of an anastomotic device inserted into vessel supporting results described herein according to an exemplary embodiment of the present disclosure.

FIG. 8 (includes FIGS. 8A-8D) shows preferred device and anchor designs.

FIG. 9 (includes FIGS. 9A-9D) shows preferred anchor designs.

FIG. 10 (includes FIGS. 10A-10D) shows additional views of preferred anchor configurations.

FIG. 11 shows a preferred device and anchor arrangement.

FIG. 12 (includes (FIGS. 12A-12C) shows additional preferred devices of varying lengths.

FIG. 13 (includes (FIGS. 13A-13F) and FIG. 14 (includes FIGS. 14A, 14A′, 14B, 14B′, 14C, 14C′, 14D, 14F, 14G, 14H, 14I, 14J, 14K, 14L, 14M, 14N, 14O, 14P) show selected anchor arrangements and coupling devices.

FIGS. 15 and 16 show device application systems.

FIG. 17 shows exemplary composite device systems.

FIG. 18 is a photograph of further composite device systems.

FIG. 19 (includes FIGS. 19A-19C) shows a device manipulation tool.

FIG. 20 (includes FIGS. 20A-20C) shows a further device manipulation tool.

FIG. 21 (includes FIGS. 21A-21C) shows a further device manipulation tool.

FIG. 22 (includes FIGS. 22A and 22B) shows additional preferred coupler devices.

FIG. 23 depicts a present coupler positioned within a vessel.

FIG. 24 (includes FIGS. 24A and 24B) shows an extended connector device joining two spaced vessels (FIG. 24A and in a bypass procedure (FIG. 24B).

FIG. 25 shows a further extended connector device system.

FIG. 26 shows a further extended connector device system.

FIG. 27 (includes FIGS. 27A-27C) depicts a preferred device and use of Example 1 which follows.

FIG. 28 (includes FIGS. 2A-28D) depicts testing and use of a preferred device of Example 2 which follows.

FIG. 29 (includes FIG. 29-29C) depicts in vivo use of a preferred device of Example 3 which follows.

FIG. 30 (includes FIGS. 30A-30B) depicts in vivo use of a preferred device of Example 4 which follows.

It should be understood that the appended drawings are not necessarily to scale, presenting a somewhat simplified representation of various features illustrative of the basic principles of the disclosure. The specific design features of the present disclosure as disclosed herein, including, for example, specific dimensions, orientations, locations, and shapes will be determined in part by the particular intended application and use environment.

### DETAILED DESCRIPTION

As discussed, we now provide new coupler devices that suitably may comprise: a member, having a passageway, wherein upon insertion, the member can engage with the vessel. Suitably, the member may be a tubular member.

Preferred coupler devices comprise a member that comprises one or more protrusion, and preferably the protrusions engage a vessel during use. In one aspect, suitably the member includes one or more protrusions on a surface of at least one end of the member. In one aspect, suitably the one or more protrusions are present on an outer surface of the member. In another aspect, suitably the one or more protrusions are present on an inner surface of the member. In a preferred aspect, the member includes a plurality of protrusions positioned on each end of the member.

The protrusions may be of a variety of configurations. In preferred aspects, the member comprises protrusions having a substantially triangular or wedge shape, including protrusions that have a substantially acute triangular shape.

Certain preferred systems may comprise a rigid or flexible linkage that separates a plurality of protrusions positioned on at least one end of the device member.

In certain aspects, the device member comprises a plurality of protrusions positioned on each end of the tube and each end plurality are separated by at least 2 cm. In other aspects, the device members comprises a plurality of protrusions positioned on each end of the tube and each end plurality are separated by at least 4, 6, 8 or 10 cm.

In certain preferred aspects, the device member comprises a plurality of protrusions that extend vertically at an acute angle from the member planar surface. In one embodiment, the device member comprises 1) a first plurality of protrusions that extend vertically at an acute angle from the member planar surface and 2) a second plurality of protrusions that extend vertically at an acute angle from the member planar surface.

In certain preferred devices, the member comprises 1) first plurality of protrusions are positioned on a first end of the member and 2) a second plurality of protrusions are positioned on a second end of the member.

In additional preferred aspects, a device member may comprise a plurality of protrusions that have a unidirectional orientation.

In a further preferred aspect, a device member may comprise a plurality of protrusions that have a bidirectional or multidirectional orientation.

In various aspects, protrusions of a device may vary in a variety of characteristics, including material of construction, size, frequency and/or orientation, and/or shape and/or arrnagement. For instance, in one aspect, a device member may comprise a plurality of protrusions that have substantially the same vertical height. In another aspect, a device member may comprise a plurality of protrusions that have differing vertical height. In a further aspect, a device member may comprise a plurality of protrusions that are composed of substantially the same material. In a yet further aspect, a device member may comprise a plurality of protrusions that are composed of differing material.

The presently disclosed subject matter will be described more fully herein after with reference to the accompanying drawings, in which some, but not all embodiments of the inventions are shown. The presently disclosed subject matter may be embodied in many different forms and should not be construed as limited to the embodiments set forth herein; rather these exemplary embodiments are provided so that this disclosure will satisfy applicable legal requirements. Indeed, many modifications and other exemplary embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not limited to the specific embodiments disclosed and that modifications and other exemplary embodiments are intended to be included within the scope of the appended claims.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

An aspect of the present disclosure a sutureless anastomotic device or coupler that simplifies that surgical procedure of connecting multiple vessels. The device provides sufficient support during the healing process of the vessel, artery, or the like. By eliminating the need for a suturing process to join the vessels, a surgical procedure is simplified and accomplished in less time. Additionally, the anastomotic device minimizes the damage to vessels, requires less healing time, and reduces the pain or complications to patients. As discussed, the anastomotic device described herein is useful across various surgical disciplines including, but not limited to, vascular surgery, plastic and reconstructive surgery, oral and maxillofacial surgery, neurosurgery, ophthalmology, urology, bowel surgery, interventional radiology, and the like. The device in various configurations including varying cross-sectional dimensions may be used in surgical applications of a micro size up to a macro size. The device is also not limited to connected vessels within the body. Thus, the device may also be used to connect an interior vessel to an exterior vessel. For example, the device may connect an interior tubular structure with an exterior colonoscopy bag or other type of exterior vessel.

Additionally, the anastomotic device described herein may be manufactured using three-dimensional (3D) printing thus allowing for micro-sized devices to be manufactured and increasing the applicability of the device across multiple disciplines and providing patient-tailored sizing. This technique allows for high resolution as well as rapid and efficient printing thus reducing overall manufacturing waste. Notably, the manufacturing process is not limited to 3D printing and other techniques may be used such as, injection molding, vacuum molding, machining and the like.

Reference will now be made to the various drawings to describe an exemplary embodiment of the present disclosure. Notably, the anastomotic device may also be referred to herein as a stent, coupler, or microcoupler.

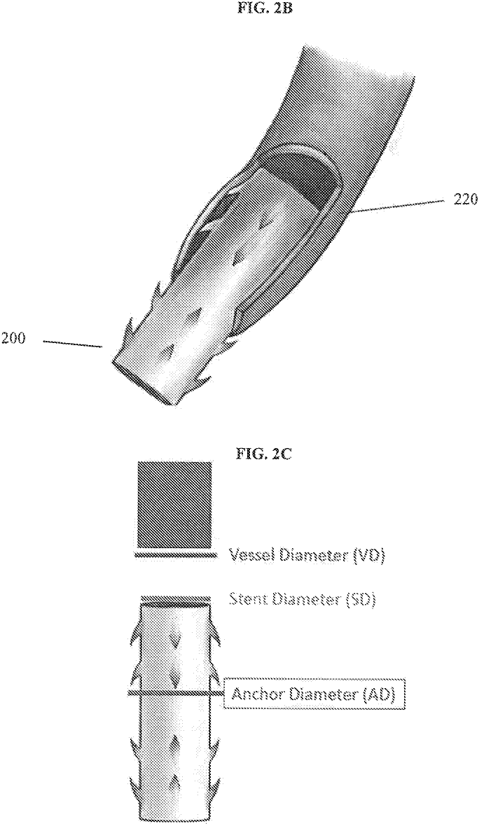

Referring now to FIG. 2A, the anastomotic device 200 according to the present disclosure may include a tube 205 having a passageway 210 formed therethrough. The tube may be formed as a cylindrical shape but is not limited thereto. Notably, the passageway 210 is formed to be hollow through the entire length thereof. Additionally, a plurality of protrusions 215 or anchors may extend from a peripheral surface of each end of the tube 205. These protrusions 215 may be formed to be opposing unidirectional barbs. A further description of the protrusions will be provided herein below.

At least one end of the tube may be inserted into a vessel, artery, veing or the like and the protrusions then engage with the inner walls of the vessel which locks the anastomotic device or stent in place. In particular, as shown in FIG. 2B, the direction of insertion of the device is oriented to the direction of the protrusions. In other words, the device is capable of smoothly entering the vessel or other tubular structure without any obstruction from the protrusions. Once inserted, any movement of the device in the opposite direction of insertion will cause the protrusion to engage with the inner wall of the vessel. Thus, the protrusions provide traction and minimize the migration of the device once the vessels are connected.

As further shown in FIGS. 2A-2B, in an exemplary system, the protrusions at a first end of the tube extend in an opposing direction to the protrusions formed at a second end of the tube. This allows for smooth insertion into respective vessels and prevents separation from two joined tubular structures (e.g., lumens) connected via the anastomotic device.

Referring to FIG. 2C, the anastomotic device may be formed in various sizes to accommodate different surgical applications. For example, a microsurgical application requires a smaller sized anastomotic device than a vascular surgery application. FIG. 2C provides an illustration of the anchor diameter (AD) of the tube, the stent diameter (SD) (e.g., diameter of the anastomotic device), as well as the diameter of the vessel (VD). Notably, the device may be formed with any number of protrusions extending from the peripheral surface thereof. Additionally, each end of the tube may include a single or multiple rows of protrusions. A distance between an end of the tube and the start of the protrusion formations may be in a range of 0 to a mid-length of the length of the coupler. For example, the protrusions on a first end of the tube may be formed starting at that first end or at any point to the middle of the tube length at which point the protrusions on a second end of the tube in an opposing direction may start.

For example, 1 to 20 rows of protrusions may be formed on each end of the tube and 1 to 20 protrusions may be formed in each row, but the present disclosure is not limited thereto. The number of rows of protrusions may vary depending on the protrusion size, the length of the tube, and the like. The inner diameter of the tube may be formed in a range of about 0.1 mm to 20 mm. In a microsurgical application, the diameter of the anastomotic device (SD) may be less than about 0.8 mm up to about 3, 4 or 5 mm. In vascular surgery, the diameter of the anastomotic device (SD) may be about 5 mm to 8 mm. In veterinary applications, the diameter of the anastomotic device (SD) may be about 0.1 mm to 20 mm. In a larger application, such as a bowel surgery, the inner diameter may be about 1 cm to 10 cm.

The sizing of the anastomotic device is important to ensure a sufficient fit between the device and the vessel as well as to ensure that there is sufficient joint tension for the anastomotic device to lock into the vessel. In particular, the sizing of the anastomotic device should be formed such that the fit between the device and the vessels (e.g., vascular wall of the tubular structure) eliminates any dead space therebetween. The following dimension ratios are merely exemplary, and the present disclosure is not limited thereto:
3 or 4 protrusions: 1.5VD > AD > VD
2 protrusions: 1.5VD = AD > VD

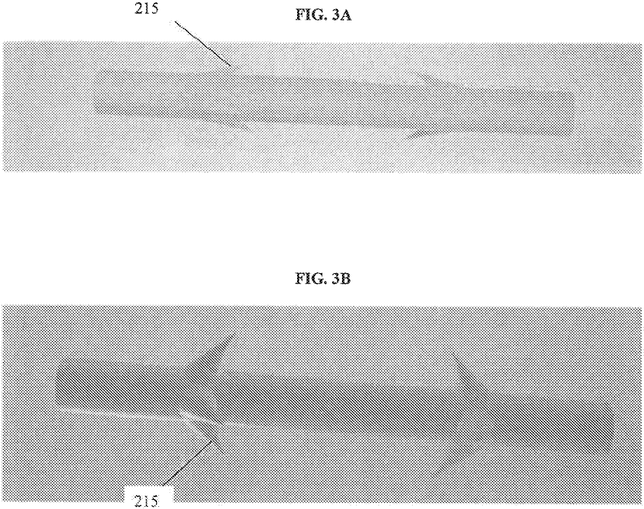

As shown in FIGS. 3A and 3B, the degree of the protrusions 215 relative to the peripheral surface of the tube may also be varied. For example, the protrusions 215 may extend at an angle ranging from 1 degree to 90 degrees from the peripheral surface of the tube. Notably, each row of protrusions is not limited to have a same protrusion angle and the protrusions may be formed with different angles. With a 1-degree protrusion angle, the protrusion may be almost in line with the baseline of the tube. In this configuration, the protrusions may still be formed to deploy with tension. FIG. 3A illustrates an example of the protrusions 215 extending 15 degrees from the peripheral surface of the tube and FIG. 3B illustrates an exemplary of the protrusions 215 extending 40 degrees from the peripheral surface of the tube. The different protrusion degrees provide for different engagement with the inner walls of a tubular structure and may be varied based on surgical application. Notably, the engagement with the inner walls of the tubular structure prevents both lateral and circumferential movement of the device. In other words, due to the engagement of the protrusions with the inner walls of the vessels, the device is prevented or inhibited from both rotating and sliding further into or out of the vessels.

In addition to forming the protrusions with different angles, the protrusions may also be formed with varying length of extension from the peripheral surface of the tube. For example, each row of protrusions may extend at a different length. Notably, the protrusions are not limited to any particular shape and may be formed with different geometries such as curved, straight, cone-shaped, tetrahedron, or the like as long as the protrusions provide friction on contact with the inner surfaces of the vessels.

According to another exemplary embodiment, the device may be formed of an expandable material. For example, upon insertion of the device into the vessels, the device may expand due to a change of stimuli including particular temperature conditions (e.g., an increase in temperature), pressure conditions, force due to the flow within the vessels, light, pH, magnetic field, or the like. Such a configuration may omit the formation of protrusions and provides sufficient tension between the device and vessels to prevent movement therebetween thus preventing potential inflammation. Alternately, the protrusions may be deployed manually when a user applies force to a lever of the device.

In another preferred configuration, a coupling device may be expandable along its length, for example to provide a telescoping system. A user then would be able to adjust the device to desired lengths.

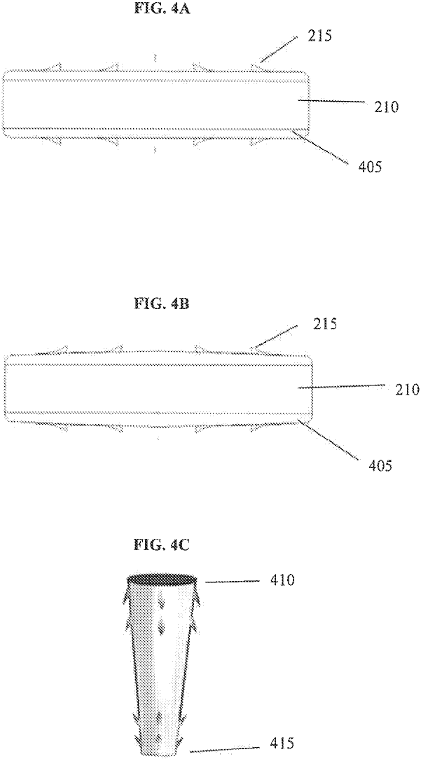

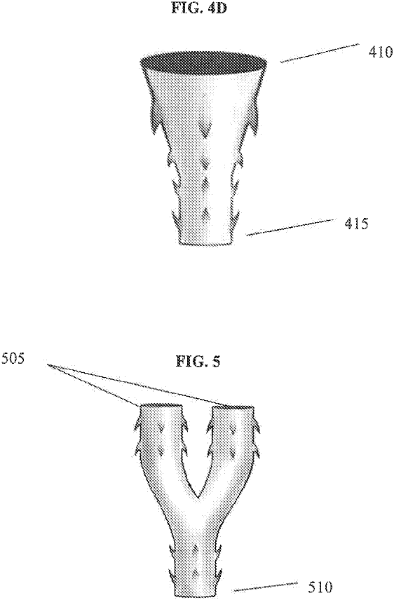

The anastomotic device of the present disclosure is not limited to a uniform cylindrical shape. Additionally, the anastomotic device is not limited to a solid cylindrical shape and may be formed foe example as a mesh-like cylinder, fenestrated, scaffolded or as a porous body, among others. The device may also be formed to be collapsible/expandable. FIGS. 4A-4D illustrate various examples of alternative structural configurations. In particular, FIG. 4A illustrates the anastomotic device with the passageway 210 of the tube having a uniform or straight shape as well as an outer surface 405 of the tube having a uniform or straight shape. This design is used in the above description of the anastomotic device. In another exemplary embodiment as shown in FIG. 4B, the passageway 210 may be formed to have a uniform or straight shape while the outer surface 405 of the tube is tapered. That is, the diameter towards the center of the tube may be greater than the diameter at each end of the tube while the inner passageway remains uniform.

FIGS. 4C and 4D illustrate configurations in which the entire shape of the anastomotic device varies. For example, in FIG. 4C, the diameter of the tube may gradually increase from a first end 415 of the tube to a second end 410 of the tube. In an alternate embodiment as shown in FIG. 4D, the second end 410 of the tube may have a diameter that is greater than the first end 415 of the tube. As shown, the diameter may remain uniform to a particular point and then increase towards the second end. The varied sizing of the ends of the tube advantageously facilitate anastomosis of different sizes. Notably, the present disclosure is not limited to the particular configuration of the first end and second end and it should be understood that the described configuration may be applicable to either end of the tube. As discussed, in another exemplary embodiment, the anastomotic device may be formed in a telescoping manner. In other words, the tube of the device may expand in length. Accordingly, the tube may be slowly advanced over time either into one vessel or both vessels. Alternately, the telescoping configuration or origami folds allows for at least one end to be advanced to a desired position upon insertion into the vessels.

In addition the to the one-to-one coupler device described above, the anastomotic device may also be formed to connect more than two vessels. As shown in FIG. 5, a second end 505 of the tube may be formed as branching outputs. That is, the second end 505 of the tube may be formed with two or more outlets in communication with the first end 510 of the tube. This anastomotic device may also be formed with each end having different sizes or each outlet 505 having a different size. The branched configuration shown in FIG. 5 advantageously allows for a vessel to be anastomosed with multiple vessels. Notably, the present disclosure is not limited to having one end formed in a branched configuration and includes a structure in which both ends are formed in a branched configuration.

The anastomotic device of the present disclosure may be formed of a flexible biocompatible, biostable, or biodegradable material, but is not limited thereto. For example, the device may be produced from Polylactide Acid (PLA), Polycaprolactone (PCL), polyurethane (PU), polyether ether ketone (PEEK), polyethylene terephthalate (PET), or a combination thereof. A preferred polyether ketone material is VESTAKEEP® PEEK from Evonik. These materials provide sufficient flexibility and rigidity to achieve a secure joint connection. Additionally, the materials improve long-term patency by providing support for blood vessels during the time it takes for the vessels to heal. The device may also be produced with or without coatings. For example, the device may be coated with pharmacologic or chemical agents to enhance vascular regeneration and prevent complications such an endothelial proliferation and thrombosis, or coated with materials for visualization. Notably, the present disclosure is not limited to the above materials and may further include other materials such as colored materials, radiopaque materials, radio opaque dopants, radiotraced materials capable of tracking decay rate and position of the device, translucent to opaque materials, or the like. Additionally, the protrusions themselves may be made of different materials or with different physical, chemical and/or biomedical characteristics than the tube of the anastomotic device.

Additional preferred materials of construction of the present coupling devices includes a polycarbonate such as Lexan® Copolymer LUX9130T; a polyurethane such as Tecoflex®, Carbothane®, Pellethane®, and/or Tecothane® (Lubrizol); PTFE (polytetrafluorethylene) and/or a eptfe such as e-ptfe available from International Polymer Engineering, Tempe, AZ.

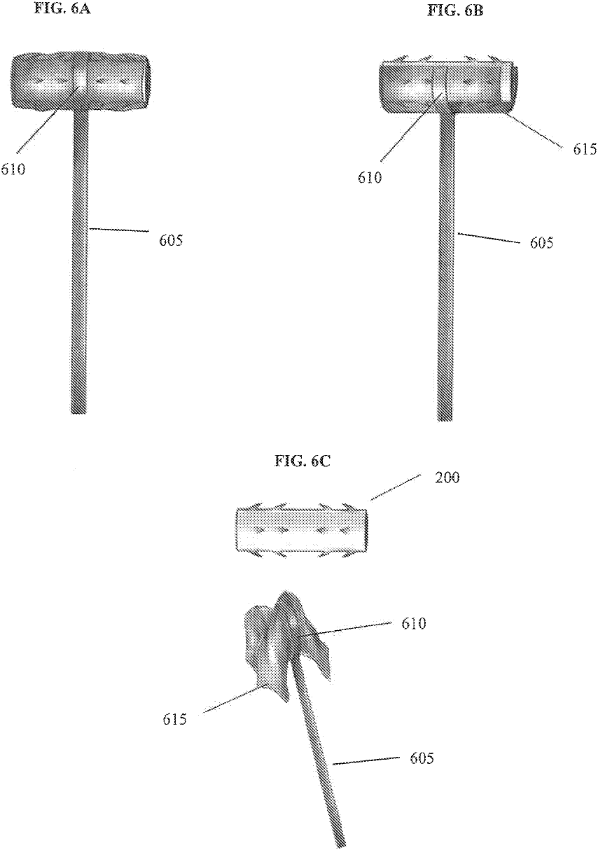

Moreover, another aspect of the present disclosure provide an anastomotic system that further includes a manipulator 605 as shown in FIG. 6A. The manipulator may be used to facilitate the insertion of the anastomotic device into the vessel. As shown, a distal end of the manipulator 605 may include a clamp 610 (e.g. claw or other gripping mechanism) that grasps the tube. In particular, as shown in FIG. 6B, the clamp 610 may circumferentially grasp the tube to deploy the device. The tube may thus be held by the manipulator 605 as a first end of tube is inserted into a first vessel and then a second end of the tube is inserted into a second vessel.

Once inserted, the clamp is able to be released to thus secure the anastomotic device between the vessels. The use of the manipulator allows for the anastomotic device to be manipulated into an optimal position without yet engaging the protrusions into the inner walls of the vessel.

As further shown in FIGS. 6B and 6C, the manipulator may include a cover 615 that is attached to the clamp 610. The cover 615 covers and protects the protrusions 215 during insertion of the tube into the first and second vessels. This further prevents the protrusions from prematurely engaging with the inner walls of the vessels and facilitates proper alignment prior to deployment of the protrusions. Once inserted and positioned, as described above, the clamp may be opened to release this tube. This release also removes the cover 615 attached to the clamp 610 thus allowing for deployment of the protrusions into the inner walls of the vessels. Additionally, the process of removing the clamp and pulling the protective cover results in a central force which causes the ends of the vessels to be pulled together thus further securing or locking the anastomotic device in place.

According to another aspect of the present disclosure, an anastomotic coupling method is provided in which the anastomotic device described above is operated. In particular, the method includes first grasping, by a clamp, a tube having a passageway formed therethrough and a plurality of protrusions extending from a peripheral surface of each end of the tube. A first end of the tube may then be inserted into a first vessel to engage the protrusions formed thereon with inner walls of the first vessel. A second end of the tube may be inserted into a second vessel to engage the protrusions formed thereon with inner walls of the second vessels. The tube may then be released from the clamp to join the first and second vessels.

Additionally, as described above, a cover may further be attached to the clamp to cover the protrusions during insertion into the vessel. The clamp may then be released to also remove the cover together with the clamp and expose the tube into the vessels thus causing the protrusions to engage with the inner walls of the vessels. The protrusions at each end of the tube may be formed in opposing directions to thus prohibit the separation of each end of the tube from the respective vessels and secure or lock the device between the vessels. In other words, as the tube is pulled out from the vessel, the protrusions engage further into the inner walls of the vessel, thus blocking the movement of the tube. Optionally, as a last step, the method may include a pauci-suturing step. In particular, this process includes adding safety stitches over the joined vessels. Notably, this pauci-suturing process is different from requiring a complete manual suturing for joining the vessels.

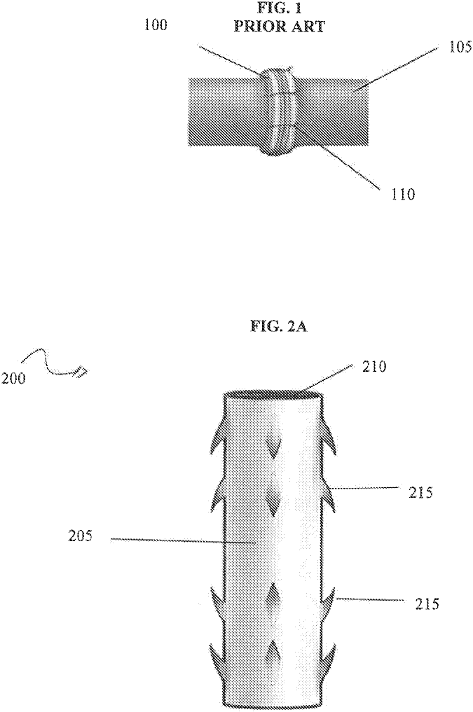

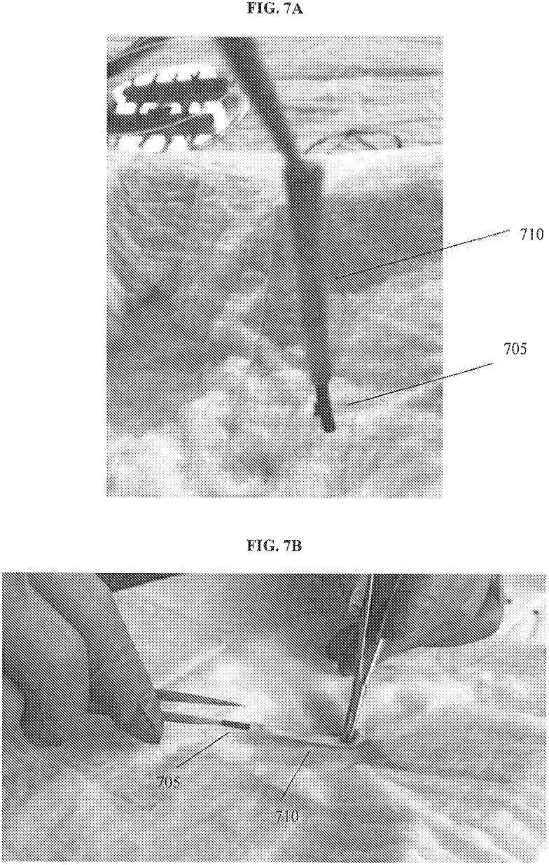

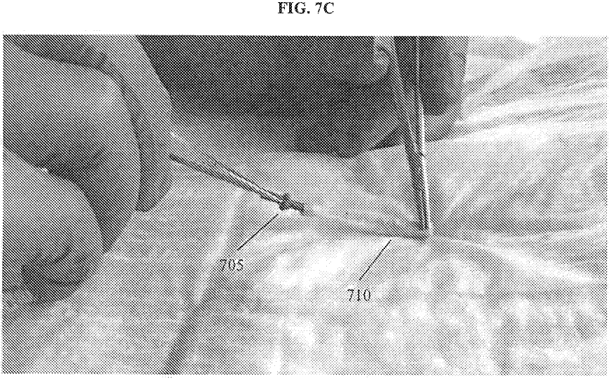

FIGS. 7A-7C provide a view of an anastomotic device inserted into a pig vessel to show the advantageous results of the present disclosure. In particular, FIGS. 7A-7C show an anastomotic device 705 inserted into a pig vessel 710 without requiring a suturing process. The figures further show that the protrusions on the device prevent the device from disengaging from the vessel. That is, the figures provdiametere results of various tests including a gravity test, an engagement test, and a lock test. Notably, prior devices have failed each of these tests. For example, the conventional device shown in FIG. 1 would fail the gravity test. The gravity test of FIG. 7A shows that the device 705 remains connected to the vessel 710 against gravity with one end of the vessel being suspended vertically (e.g., via tweezers) and the device remaining engaged in the bottom end of the vessel. Next, FIG. 7B illustrates an engagement test. In this test, the device 705 remains in place (connected to the vessel) while the vessel 710 is maneuvered (such as via tweezers as shown) without stretching the vessel beyond static length. Lastly, FIG. 7C illustrates a lock test. In this test, the device 705 and vessel 710 are pulled away (here as shown via tweezers) from each other beyond the vessel’s static length, for example an additional 10, 20 or 30 percent of force is applied to pull device 705 and vessel 710 away from each other beyond the vessel’s static length.. As shown, during such a pulling motion, the device 705 remains connected to and engaged with the vessel 710 thus locking the device into the vessel and also preventing any movement of the device. These figures thus show the locking engagement provided by preferred anastomotic devices as described herein.

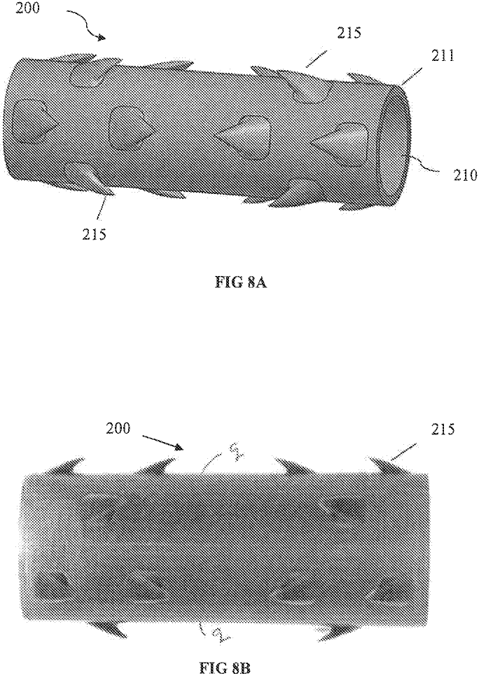

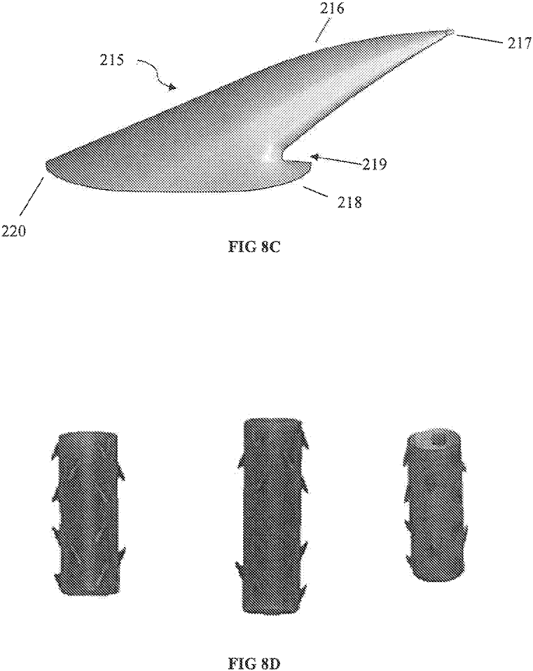

FIGS. 8A, 8B, and 8D depicts further preferred devices 200 with opening 210, device edge or terminal end 211 and anchor or protrusions 215. In FIG. 8A, end 211 is shown as forming a substantially vertical or perpendicular surface with respect to the planer surface that forms the length of device 210. Other edge 211 configurations also will be suitable, including an edge that forms an acute angle relative to the device planar surface such as the rounded edge 211 shown in FIG. 11.

FIGS. 8C, 9A-9D, 10A-10D and 11 show various preferred protrusions or anchors 215 that include vertical tip 217, adjacent bottom tip or end 218 and opposed bottom tip or end 220.

It has been found that a forward sloping anchor tip can provide particularly favorable coupling of a vessel or other tissue. In one aspect, such preferred device anchors with forward sloping tips have a triangular or an acute triangular configuration or design. Preferred device anchors also may be described as wedge-shaped, angled wedge-shaped or acute angled wedge-shaped. In a related aspect, preferred device anchors or protrusions may be tooth-shaped (with pointed end), or angled tooth-shaped, including acute angle tooth-shaped.

In certain preferred configurations, the anchor vertical forward tip (such as tip 217 shown in FIGS. 8C, 9B and 10C) may have a sloped angle of between about 10, 15, 20, 30 or 40 degrees to about 50, 60, 65, 70, 75, 80, 85 or 86, 87, 88 or 89 degrees relative to the planar surface of a device (planar surface q shown in FIG. 8B), more typically vertical forward tip may have a sloped angle of between about 35 and 80 degrees or about 40 to 75 relative to the planar surface q of a device. In certain aspects, the anchor vertical forward tip (such as tip 217 shown in FIGS. 8C, 9B and 10C) may have a sloped angle of at least 5, 10, 15, 20, 25, 30, 35, 40, 45, 50, 55, 60, 70, 75 or 80 degrees relative to the planar surface of a device (planar surface q shown in FIG. 8B). As discussed, in certain aspects the anchor vertical forward tip (such as tip 217 shown in FIGS. 8C, 9B and 10C) will have a angle of less than 90 degrees relative to the planar surface of a device (planar surface q shown in FIG. 8B).

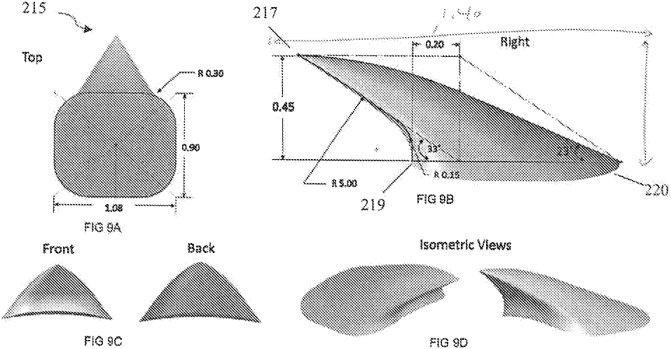

FIGS. 9A and 9B depict dimensions of one suitable system. Thus, FIG. 9A shows protrusion base of 0.90 mm by 1.08 mm. Each of those dimensions suitably may vary, for example, each base dimension shown in FIG. 9A may be the same or substantially the same or different and may be suitably e.g. 0.2, 0.3, 0.4, 0.5, 0.6, 0.7, 0.8. 0.9, 1.0, 1.1, 1.2, 1.3, 1.4, 1.5, 1.6, 1.7, 1.8, 1.9, 2.0 mm or more. Each base dimension would be the substantially the same if the dimension differs no more than 1, 2, 3, 4 or 5 percent from the other dimension of the base.

FIG. 9B shows an exemplary preferred protrusion vertical height of 0.45 mm that extends from the protrusions bottom (such as 219) to the highest point 217 of the protrusion. A protrusion vertical height suitably may vary and include for example up to 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 mm or more. FIG. 9B also shows an exemplary preferred protrusion length of 1.4 mm that extends from 217 to 220 as shown in FIG. 9B. A protrusion length suitably may vary and include for example up to 0.5, 0.6, 0.7, 0.8, 0.9, 1.0, 1.1, 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4., 4.6, 4.8, or 5.0 mm or more.

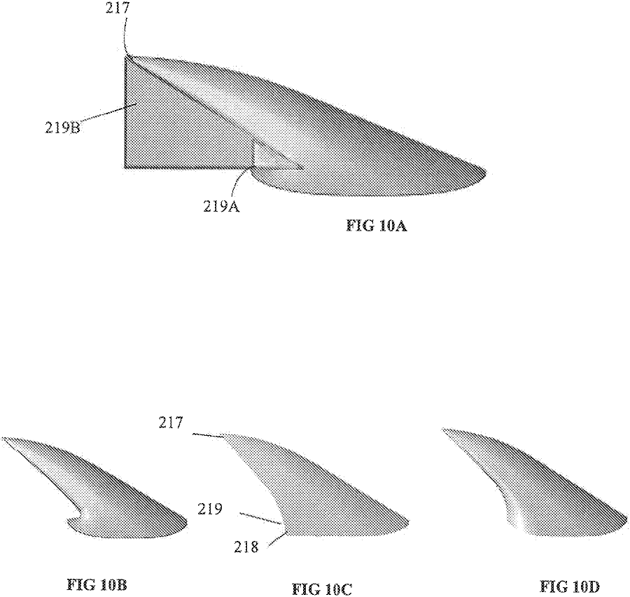

FIG. 10A depicts regions 219A and 219B which are spaces defined in part by the protrusion shown in the figure. In FIG. 10B, 219A may be a region of flow along the protrusion.

FIG. 11 shows a preferred device 210 with plurality of protrusions 215 that have a preferred acute-angled wedge shape. Right-facing protrusions 215A on device left side 210A are separated by a minimal distance y, y′ from left-facing protrusions 215B on device right side 210B. As referred to herein, distance y is the closest distance between opposing protrusions (e.g., as shown in FIG. 11, anchors 215′ and 215″ having facing distal tips 217, distinct from protrusions 215′ and 215‴ that are arranged with distal tips in substantially the same direction). As referred to herein, distance y′ is the closest distance between protrusion foot or base portions 218 of opposed positioned protrusions as generally shown in FIG. 11.

The distance y and y′ suitably may vary significantly and may be for example, up to 5, 10, 15, 20, 25, 30, 35, 40, 45, 50, 55, 60, 65, 70, 75, 80, 85, 90, 95 or 100 mm or more, such as up to 150, 200, 250, 300, 250, 400, 450, 500, 550, 600, 650, 700, 750, 800, 850, 900, 950, 1000, 1050, 1110, 1150, 1200, 1250, 1300 mm or more. Devices 200 configured with an extended region of length y (such as y being at least 0.25, 0.5, 0.75, 1, 1.25, 1.5, 1.7, 2, 2.25, 2.5, 2.75, 3, 3.25, 3.5, 3.75 or 4 feet) may be useful for example in bypass procedures.

As discussed, the device overall length x as well as cross-section z may vary widely. In particular, lengths y, y′ may vary widely as generally depicted in FIGS. 12A, 12B and 12C.

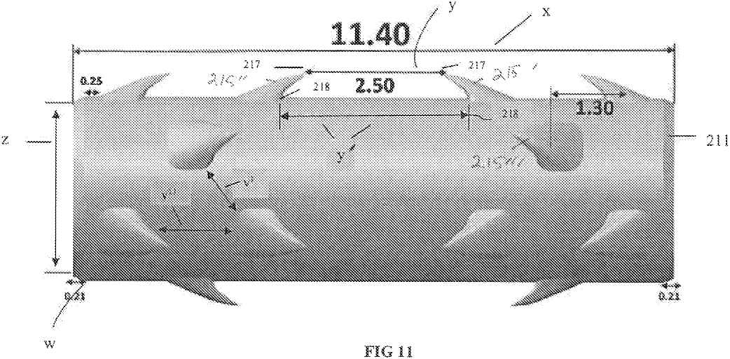

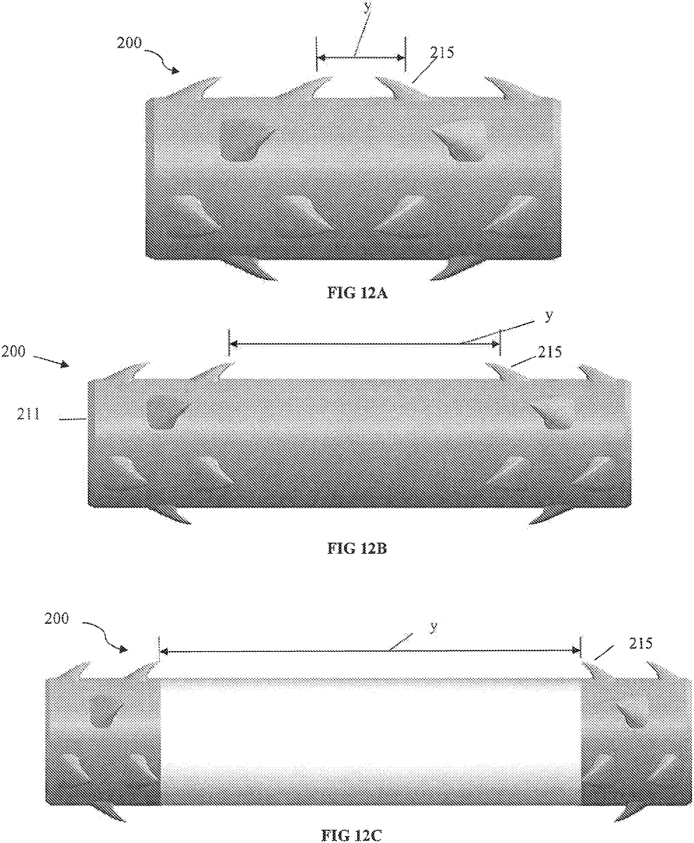

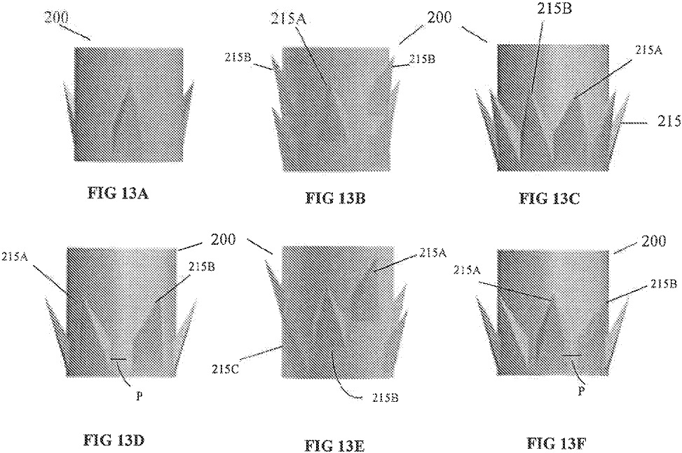

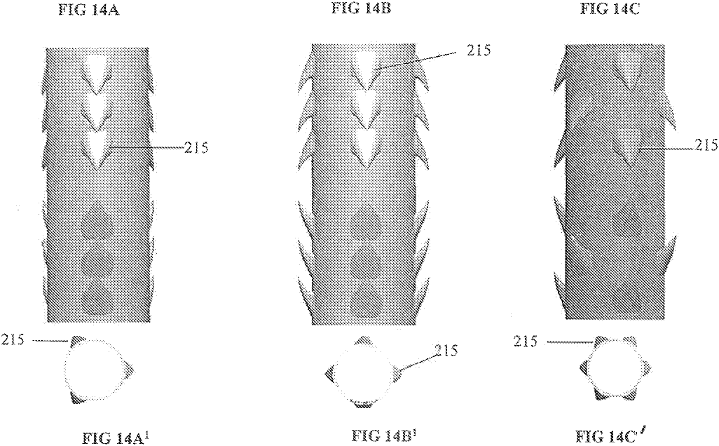

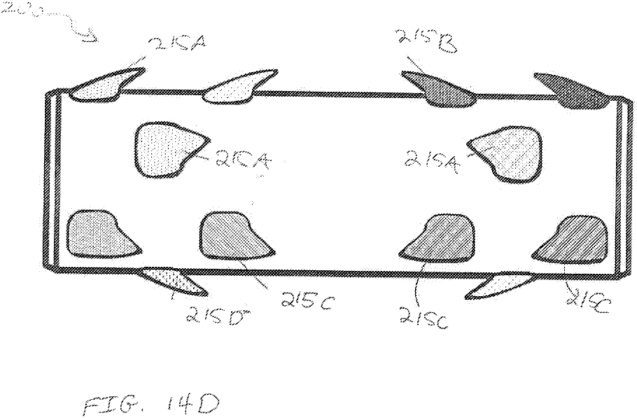

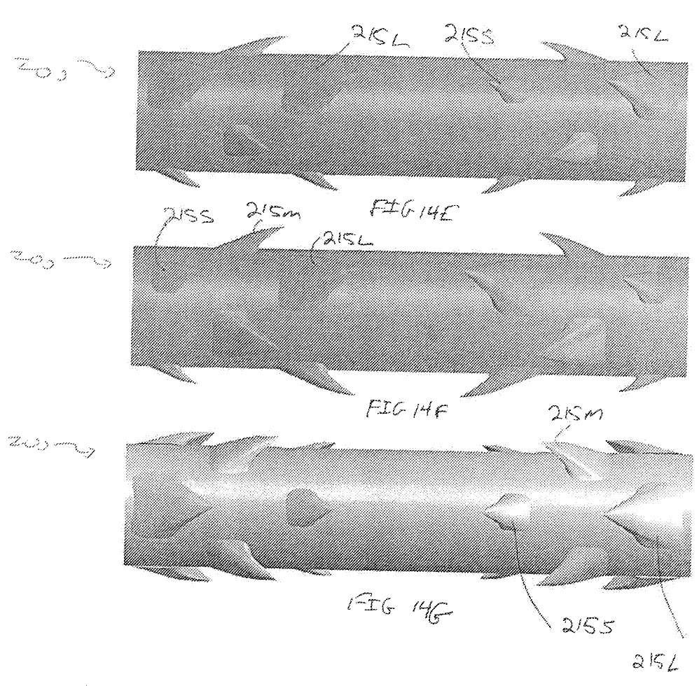

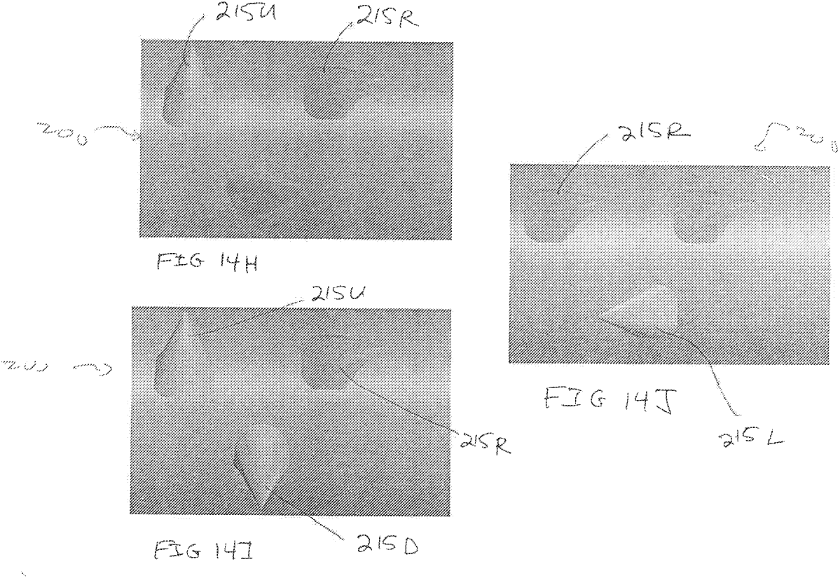

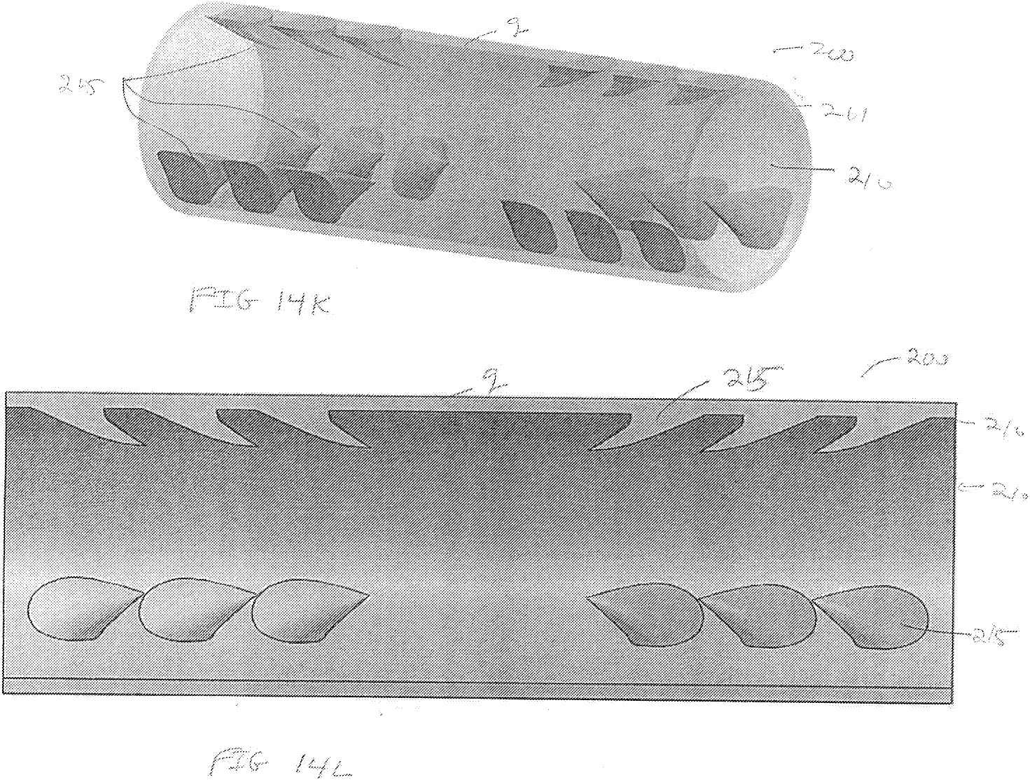

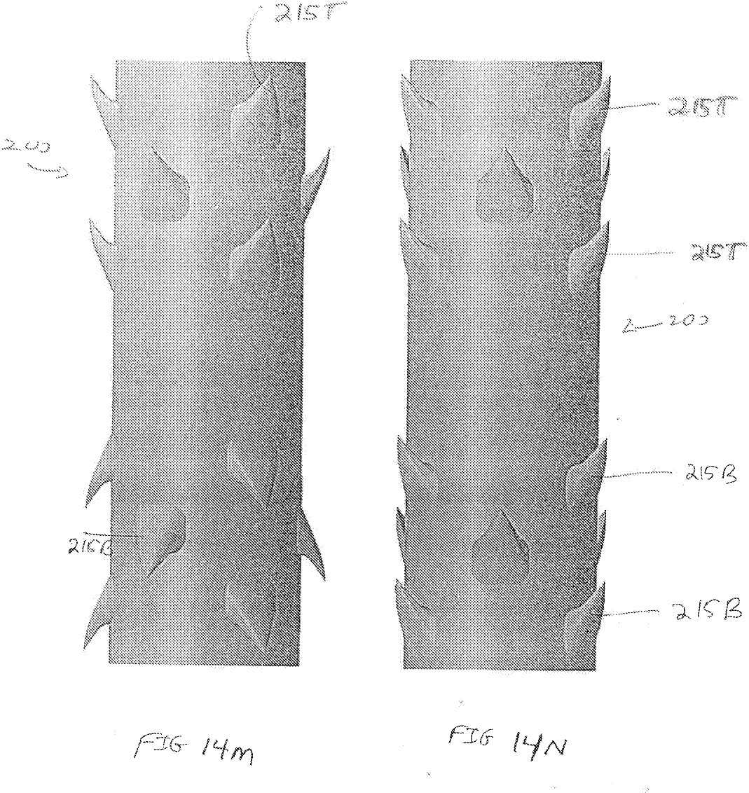

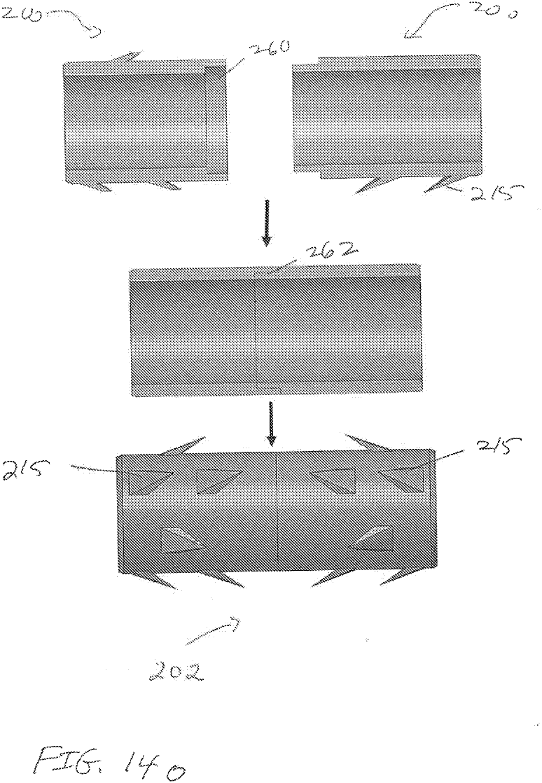

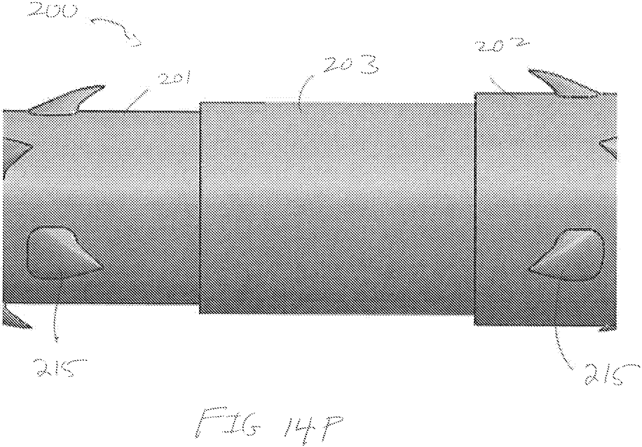

As also discussed, the arrangement and density and/or frequency of device protrusions 215 suitably may vary. FIGS. 13A through 13F depict various protrusion 215 arrangements. Thus, FIG. 13A depicts protrusions 215 arranged substantially parallel to adjacent protrusions that together circumscribe device 200. In FIG. 13A, the plurality of protrusions are spaced at approximately 90 degrees; i.e., the protrusions 215 are spaced at each quarter turn (90 degrees) of the device. The protrusion row frequency suitably may vary, including where the device has multiple rows of protrusions, or the row frequency can be greater than as depicted in FIG. 13A, such as exemplified in FIGS. 13C, 13E and 13F where the distance p between adjacent protrusions of a row is less than the quarter turn poisoning of FIG. 13A. FIG. 13C depicts an abutting protrusion arrangement (distance p is zero).

In additional preferred configurations, adjacent protrusions or anchors 215 may be spaced diagonally along the length of device 200. Thus, as exemplified in FIG. 13E, adjacent protrusions 215A, 215B and 215C are diagonally offset along the length of device 200.

FIGS. 14A, 14A′, 14B, 14B′, 14C and 14C′ further depict varying frequency of protrusions 215 along a device circumference. Thus, FIGS. 14A, 14A′ exemplify a device with three protrusionss along the device circumference, FIGS. 14B, 14B′ exemplify a device with four protrusions along the device circumference, and FIGS. 14C, 14C′ exemplify a device with six protrusions 215 (through an offset row) along the device circumference.

The present devices and anchors and protrusions suitably or preferably may be further varied including with respect to materials, protrusion orientation and protrusion shape and size, including among multiple protrusions present a single device.

For instance, as depicted in FIG. 14D, protrusions 215 on a device 200 may be of differing materials of construction, such as where depicted protrusions 215A, 215B, 215C and 215D are each composed of different materials. For example, protrusions 215A may be composed of PEEK, protrusions 215B may be composed of a polyurethane, protrusions 215C may be composed of a polycarbonate, and protrusions 215D may be composed of PTFE.

Protrusions 215 of a device 200 also suitably may vary in size, including in vertical height from the device planar surface and length and cross-section. Such size variation is exemplified by the devices 200 depicted in FIGS. 14E, 14F and 14G, where a single device 200 is shown with protrusions 215L (comparatively large size with respect to other protrusions of the single device), 215S (comparatively small size with respect to other protrusions of the single device) and 215M (comparatively intermediate size with respect to other protrusions of the single device).

Protrusions 215 of a device 200 also suitably may vary in orientation or direction. Such directional variation is exemplified by the devices 200 depicted in FIGS. 14H, 14I and 14J, where a single device 200 is shown with protrusions 215U, 215R, 215L, 215D each have different directional orientation. While the exemplary devices 200 of FIGS. 14H, 14I and 14J depict protrusions directionally offset with respect to other protrusions of the device at 90 degrees or 180 degrees, other directional variations also will be suitably, such as where protrusions of a single device (including adjacent protrusions of the device) are directionally offset by up to 10, 20, 30, 40, 50, 60, 7, 80, 85 or 89 degrees, or up to 95, 100, 110, 120,130, 140, 150, 160, 170 or 189 degrees.

As discussed, protrusions also may vary in angle that they extend from a device planar surface.

As discussed, the present coupling devices also may comprise one or more protrusions on an inner wall surface of the device, for example where the device would encase vessels being adjoined. For instance, FIGS. 14K and 14L depict devices 200 where protrusions 215 are within device opening 215 positioned on device wall q.

As also discussed, the present coupling devices also may comprise protrusions 215 where all or a substantial portion (e.g. at least 55, 60, 70, 80, 90 or 95 percent of the total protrusions of a device) are aligned in opposed or substantially opposed (i.e. substantially the opposed within up to 1, 2, 3, 4, 5, 6, 7, 8, 9 or 10 degrees) direction with protrusion tips also oriented in opposed or substantially opposed direction. This is exemplified by device 200 of FIG. 14M where protrusions 215T are in such opposed configuration with respect to protrusions 215B.

As also discussed, the present coupling devices also may comprise protrusions 215 where all or a substantial portion (e.g. at least 55, 60, 70, 80, 90 or 95 percent of the total protrusions of a device) are aligned in the same or substantially the same (i.e. substantially the same being within up to 1, 2, 3, 4, 5, 6, 7, 8, 9 or 10 degrees) direction. This is exemplified by device 200 of FIG. 14N where protrusions 215T are directionally oriented the same as protrusions 215B.

FIG. 140 exemplifies a further preferred system where multiple devices 200 with protrusions 215 may be joined such as by bridge 262 to provide the composite device 202 shown as the bottom image of FIG. 140. A joining device 200 may comprise a locking or engagement mechanism 260 to facilitate coordination of the multiple devices to form the composite device 202.

FIG. 14P exemplifies a preferred system telescoping or expandable system where device 200 comprises portions 201 and 202 that each contain protrusions 215. Those portions 201 and 202 are linked though telescoping or expanding segment 203 which can be advanced or retracted to thereby selectively adjust the length of device 200. As discussed above, such an adjustable system can be enable for example to advance the device as desired into a vessel or to a desired position once the device is engaged with a vessel. Telescoping mechanisms can be incorporated into the device 200 and include mating releasable latched engagements in one or more of 201, 202 and 203.

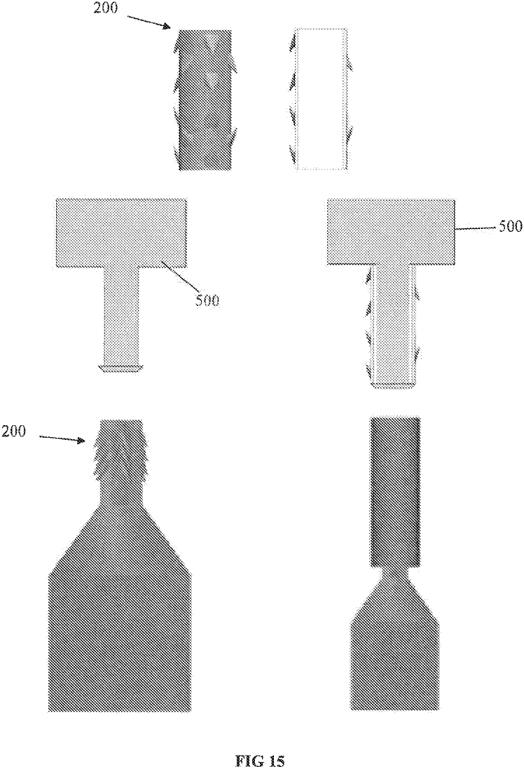

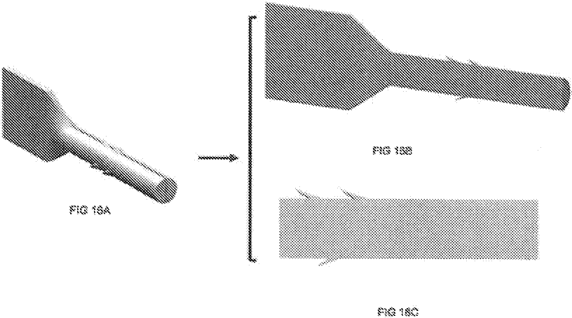

FIGS. 15 and 16 depict a further preferred system that includes a coupling device 200 together with stopper 500.

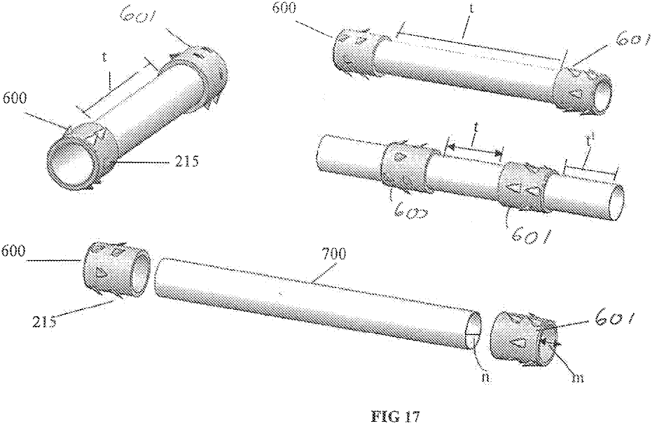

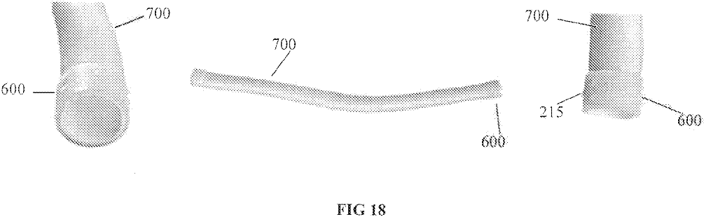

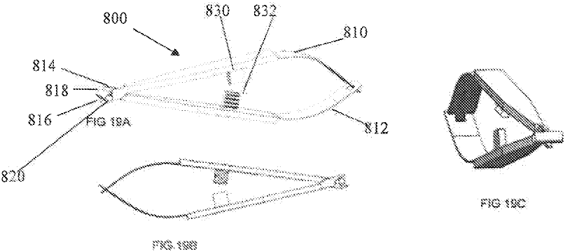

In further preferred systems, paired devices 600, 601 that each suitably contain opposing positioned anchors 215 may be utilized together with connector portion 700 onto which the paired devices 600, 601 each engage as generally depicted in FIGS. 17 and 18. Such a system can readily provide a desired spacing of opposed anchors 215 (such as a desired length y, y′ as shown in FIGS. 12A-12C, or length t as show in FIG. 17).

As depicted in FIG. 17, paired devices 600, 601 suitably each has sufficient number and arrangement of anchors 215 to securely engage a subject vessels being joined. Preferred devices 600, 601 have anchors 215 that extend vertically at acute angles with respect to the device planar sure as shown in FIG. 17, and the respective anchor distal tips 217 of devices 600, 601 are in an opposed facing configuration. Suitably the devices 600, 601 and connector 700 have a secure press fit engagement that may be further enhanced with use of an adhesive. Thus, for instance, in certain preferred systems, suitably the inner diameter m of a device 600 or 601 is no more than 1, 2, 3, 4 or 5 percent greater than the outer diameter n of a mating connector 700.

The length of connector 700 can vary widely to provide a desired extension between joined vessels, or linkage to a device such as for ex vivo treatment. For such ex vivo treatment, a single device 600 may be utilized with the opposed end of connector 700 linked to a treatment apparatus. The length of connector 700 or the length t provided between opposed linkages of connector 700 may be for example, up to 5, 10, 15, 20, 25, 30, 35, 40,, 45, 50, 55, 60, 65, 70, 75, 80, 85, 90, 95 or 100 mm or more, such as up to 150, 200, 250, 300, 250, 400, 450, 500, 550, 600, 650, 700, 750, 800, 850, 900, 950, 1000, 1050, 1110, 1150, 1200, 1250, 1300 mm or more. Devices 200 configured with an extended region of length of connector 700 (such as t being at least 0.25, 0.5, 0.75, 1, 1.25, 1.5, 1.7, 2, 2.25, 2.5, 2.75, 3, 3.25, 3.5, 3.75 or 4 feet) may be useful for example in bypass procedures.

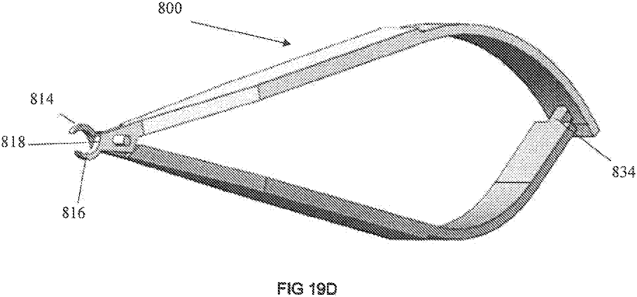

FIGS. 19-21 show manipulation apparatus that can facilitate use and placement of a coupling device.

Thus, clamping apparatus 800 depicted in FIGS. 19A-19D includes activator levers 810, 812 that can actuate grasping elements 814, 816 that engage a coupling device within space 818. In use, apparatus 800 can be held in a single hand of a medical professional and opposing forces applied to levers 810, 812 to grasp or release a coupling device positioned within space 818. Elements 830, 832 may engage to provide a reliable lock of apparatus 800. Element 834 provides optional engagement of proximal ends of levers 810, 812.

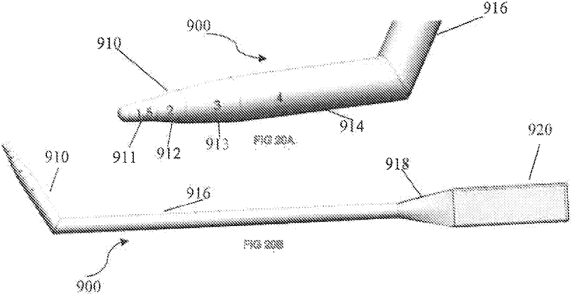

FIGS. 20A and 20B depict a further preferred system that includes sizing and applicator device 900 that includes application end 900 with sizing portions 911, 912, 913, 914 and arm and handle portions 916, 918 and 920. As shown in FIG. 20C, coupling device 200 can be positioned on end 910 and reside at regions 911, 912, 913, 914 corresponding to the coupling device size. The device 200 then can be advanced to a target vessel. Upon application of the device, device end 900 also can effectively dilate a vessel.

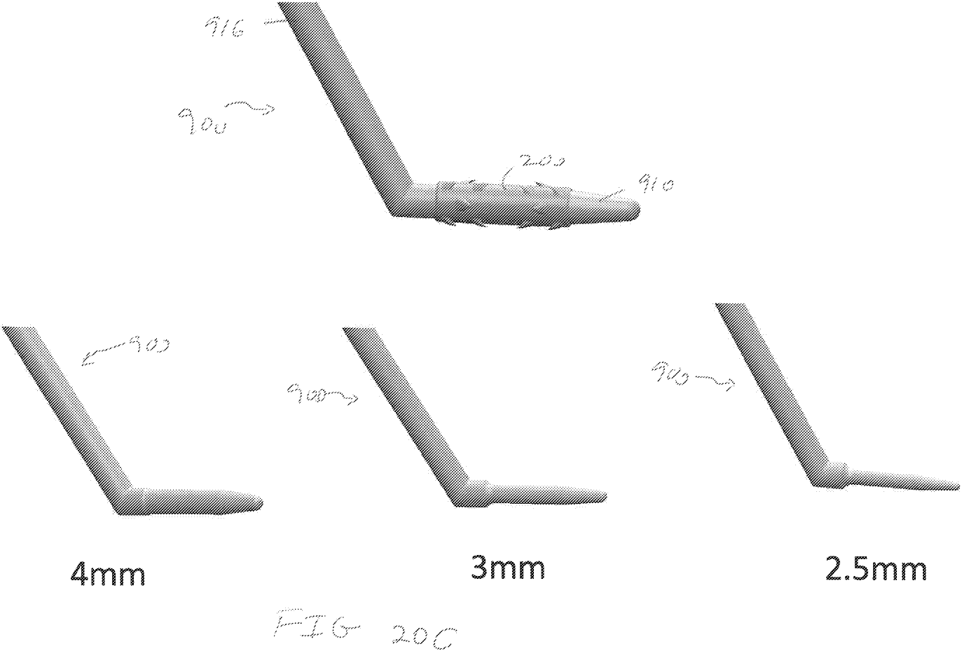

An additional clamping apparatus 960 exemplified by the apparatus depicted in FIGS. 21A-21C includes arms 968, 970 with grasping elements 964 and 966. In use, a coupling device is engaged within elements 964, 966.

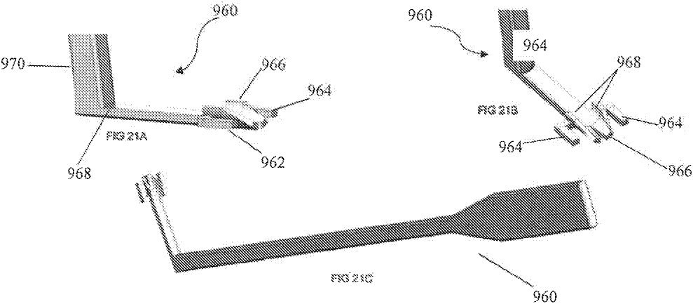

FIGS. 22A and 22B show further device configurations including the multi-branch device 200 of FIG. 22A where protrusions 215 are present on each device branch or arm 216. FIG. 22B shows device 200 that includes anchoring portions 218 with protrusions 215.

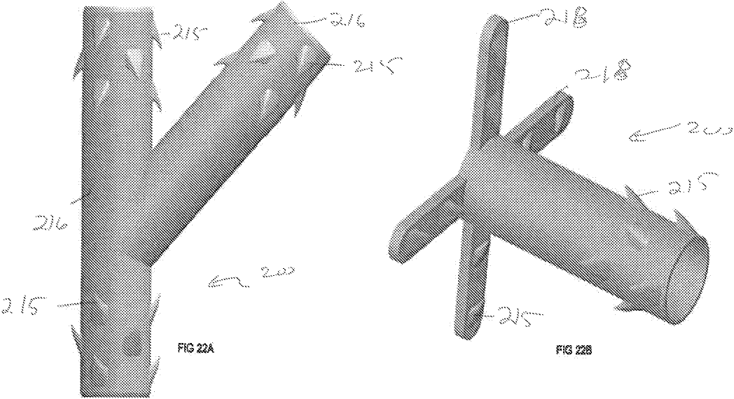

FIG. 23 depicts device 200 with protrusions 215 encased within vessel 300. Protrusions 215 suitably may abut or otherwise adhere to vessel walls 310.

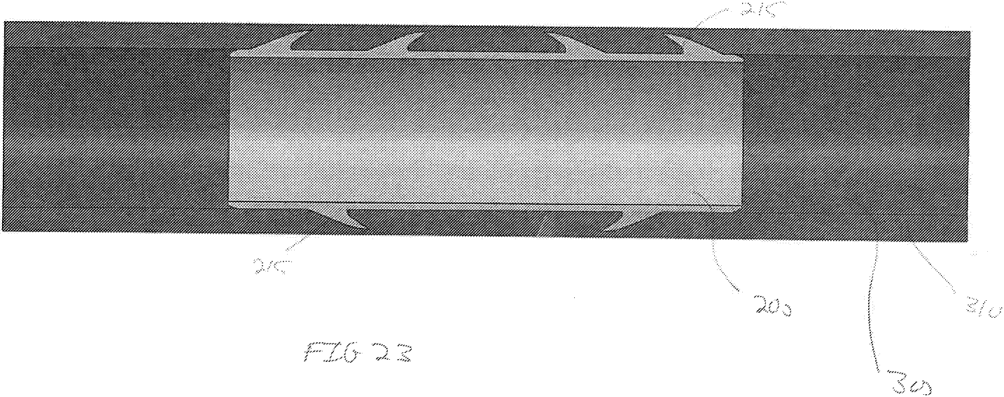

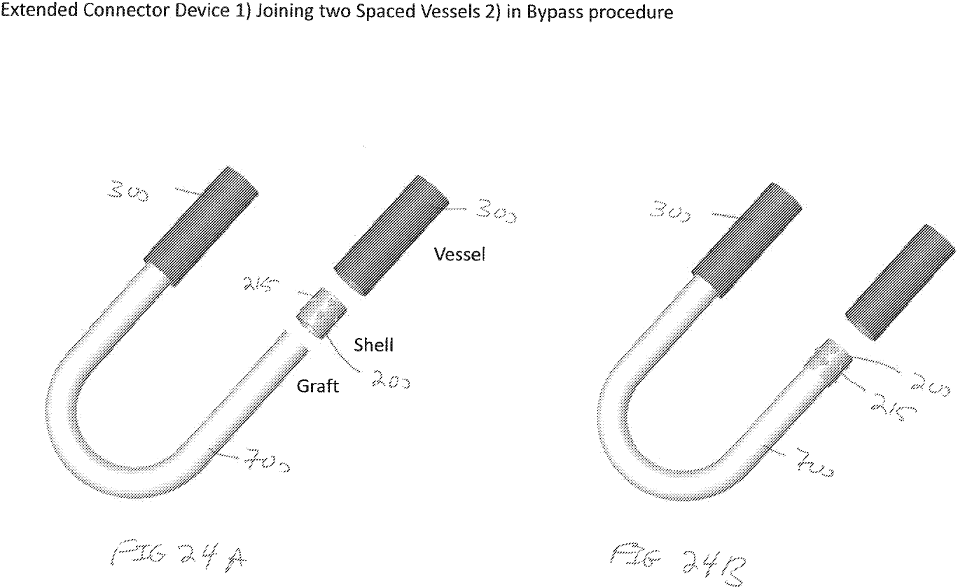

FIGS. 24-26 show further preferred systems with one or more devices 200 and extended connecting linkage 700. Thus, device 200 with protrusions 215 may be connected with linkage 700 to join spaced vessels 300 as shown in FIG. 24A. FIG. 24B depicts device 200 with protrusions 215 connected with linkage 700 in a bypass procedure. FIG. 25 depicts device 200 with protrusions 215 and linkage 700 in phantom view encased within adjoined vessel 300.

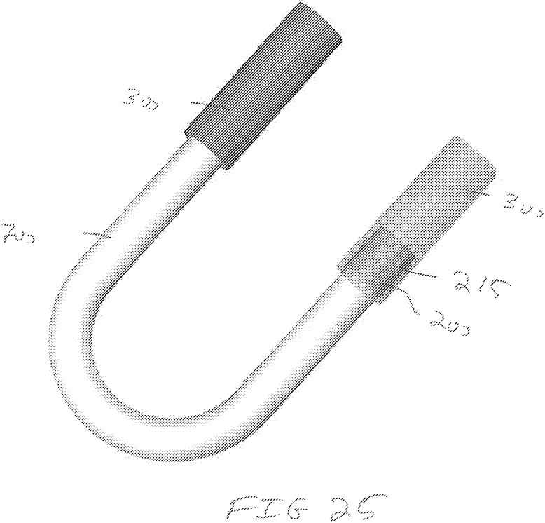

FIG. 26 shows a system with device 200 on each end of linkage 700 and encased within adjoining vessels or connectors 300 and 340.

As discussed, the present coupling devices can be used in a wide variety of procedures and provide significant advantages.

As one specific example, the present coupling may be used in procedure involving deep inferior epigastric perforators (DIEP) flaps. DIEP flaps are a type of breast reconstruction for women who had mastectomies following breast cancer. Skin and fat are removed from the lower abdomen, along with its blood supply, the deep inferior epigastric perforators (blood vessels). The flap is then transplanted onto the patient’s chest connecting the DIEP blood supply to blood supply in the patient’s chest. A present coupling device can be used to decrease anastomosis time for the bilateral DIEP - which can involve up to six blood vessels (one artery and two veins for each side), reducing microsurgery operative time from many hours to one hour. The flap is then shaped into a new breast.

In this regard the present coupling devices can be used for any flaps for reconstruction.

As a further example, the present coupling devices may be used in a variety of bypass producers, for instance, Coronary Artery Bypass Surgery (CABG) which is performed to bypass a blocked coronary artery in a “heart attack”. First, the saphenous vein is harvested from the patient’s leg. The vein is then anastomosed to the aorta and coronary artery, creating a “bypass” for the blood to escape the blocked artery and perfuse the heart. A present coupling device end-to-side designs would allow for rapid anastomosis of the vein graft, decreasing tissue ischemic time.

The present coupling devices also may be advanteously used in upper and lower extremity bypass procedures. A lower extremity bypass is required for patient with a blockage in one of their arteries that results in leg pain or a wound. A conduit (either vein or graft) is connected with a healthy artery at one end, tunneled around the blockage or injury, and connected to a smaller healthy artery at the other end. A present coupling device could be used to create one of both of the arterial connections.

As another example, the present coupling devices are highly useful for treatment or repair of vascular injury. Vascular injury can happen after trauma and may involve partial or complete damage to an artery or vein. Traditionally these injuries are repaired by sewing the vessel back together with sutures. A present coupling device could be used to facilitate the repair, reducing the time needed to repair the injury.

As an additional specific example, the present coupling devices may be used in procedures involving ureter injury: During urological or gynecologic surgeries, the ureters are commonly ligated, prohibiting urine to be drained from the kidney resulting in progressive kidney damage. A present coupling device can be used to quickly repair ureteral injuries, rather than hand sewing the ureters together.

The present coupling devices also can be utilized in replantation procedures. For instance, when a limb or finger has been amputated or avulsed, it can be salvaged via replantation. During replantation, many small arteries and veins must be reconnected. A present coupling device allows for the rapid anastomosis and replantation of the limb and finger. Furthermore, the small scale anastomosis (super microsurgery, <0.8 mm) rapidly achieved with a present coupling device allows for the replantation of small structures, such as distal finger, or pediatric replantation.

The present coupling devices also can be utilized in transplantation procedures. Minimizing ischemic time during organ transplantation is a critical step, including fast and reliable anastomosis of vessels perfusing the organ. Anastomosis of organ blood vessels with a present coupling device would allow for rapid transplantation, thus extending organ viability. Furthermore in veterinary sciences or biological sciences, anastomosis time prohibits the use of small mammals as (mice, rats, rabbits) for transplant studies. Use of a present coupling device would decrease this time, allowing for small mammals to be used for research studies, rather than the current large mammal status quo (swine, felines, canines, primates).

The anastomotic coupling device of the present disclosure provides numerous advantages. For example, as discussed herein the time required from joining vessels may be decreased based on the simplified system thus facilitating a more efficient procedure. The system also connects free vessel ends together using traction thus increasing the joint strength between the vessels and thus, the anastomotic forces approximating the vessels are independent of the healing tissue integrity. Additionally, this system is independent of assistive techniques such as manual sutures. By removing the need for any manual suturing other than for adding possible safety stitches, the learning curve for operating the device is substantially reduced and the healing time is substantially reduced. The device is also size-adaptive and may be formed as a multi-vessel adaptor. The device may be manufactured using three-dimensional printing thus increasing the variety of surgical applications for which the device may be used, but as noted above, is not limited to such a process.

### EXAMPLES

### Example 1

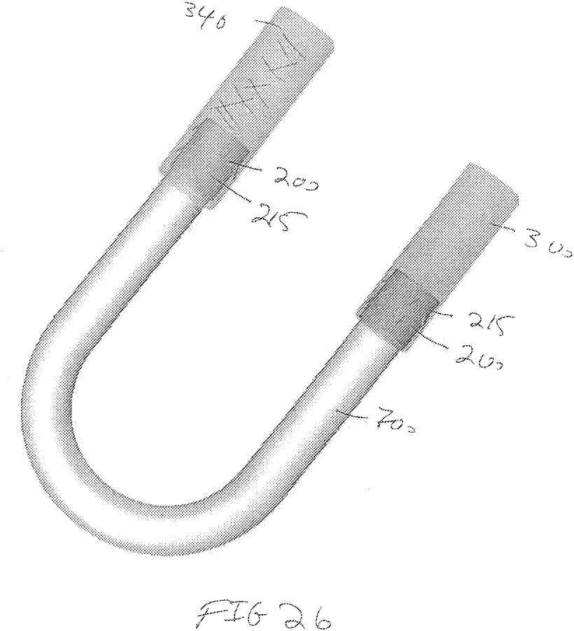

A device of the general design shown in FIG. 8A was evaluated. Light microscopy of anchor geometry and architecture of the device is shown in FIG. 27A. The device is shown in FIG. 27B adhering to tissue without trauma or penetration. As shown in FIG. 27C, traditional handsewn anastomosis versus anastomosis with the present device was evaluated in fresh ex vivo porcine carotid. The evaluation showed that the present device successfully anastomoses blood vessels, and also functions as a stent to maintain vessel patency, with minimal deformation and damage to vessel walls.

### Example 2

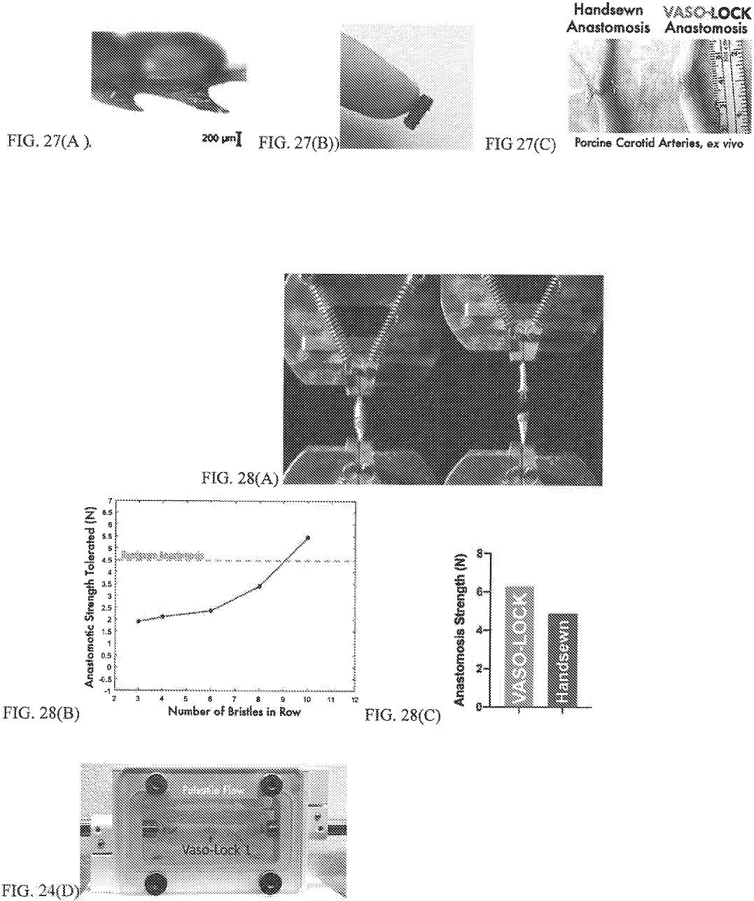

The device anchor design was optimized to a functional tolerance range of ± 0.15 mm and ± 10°; deviations from these ranges result in loss-of-function (FIG. 28A). The number of bristles and rows, arrangement, and sizes were optimized via ex vivo manual tensile strength testing with fresh cadaveric porcine carotid arteries (FIGS. 28B,C). Testing demonstrated superior force generation (6.3 N) with the present coupling device compared to handsewn anastomosis (4.9 N, FIG. 28D), with similar gross decay patterns (intimal ringing). Ex vivo pulsatile flow testing demonstrated that anastomosis with the present device tolerated physiologic flow ranges with no anastomotic leakage (FIG. 28E).

### Example 3

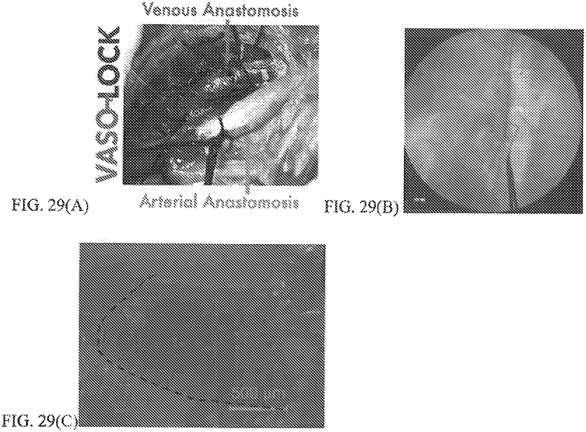

A coupling device was produced via 3D printing (3DP) using HEK resin (available from Boston Micro Fabrication). The four cut-and-repair swine models demonstrated that the present coupling device maintains femoral and iliac arterial anastomoses (FIG. 29A), with no evidence of thrombosis or leakage on angiogram within four hours of device deployment with the HEK prototypes (FIG. 29B). Transonic perivascular flow modules also demonstrated stable conservation of flow proximal and distal to device (141 mL/min), indicating negligible flow resistance across the device. Light microscopy of en bloc resected device demonstrated contact surfaces between bristles and vessel intima with no trauma or penetration (FIG. 29C).

Furthermore, an evaluated device took less than five minutes to deploy in swine vessels in the hands of both novice and experienced surgeons. Feedback received includes ease of technology adoption, procedural time reduction, and streamlined workflow requiring less personnel and resources.

### Example 4

A 3 mm i.d. coupler device corresponding to the design of FIG. 2A was put into a common femoral artery of a pig with a 4.5 mm punched out hole. The coupler was observed to cover the defect, and it was nearly hemostatic. Additional support sutures were added to hold it place and pull the hole together over it like a stent.

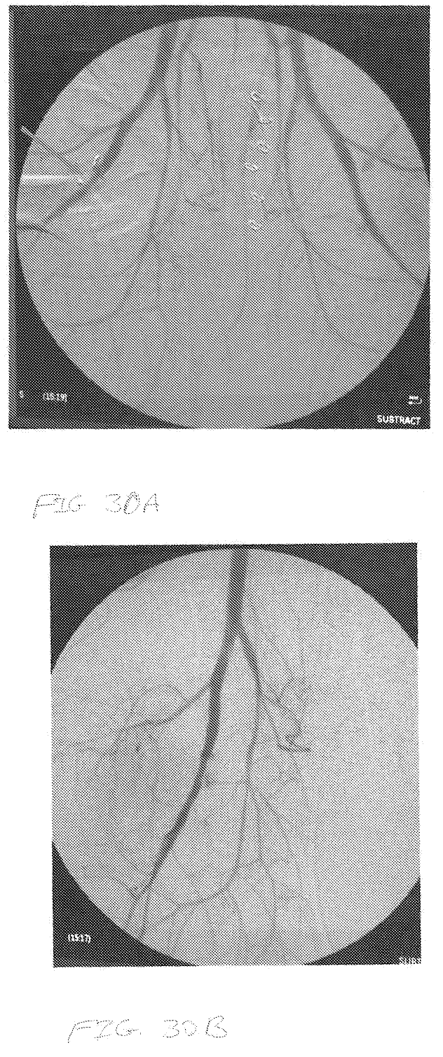

After 2 hours of introduction of the coupler, the joined artery did not thrombose. An angiogram was then taken of the right side and the image is shown in FIGS. 30A and 30B where in FIG. 30A the arrows mark the coupler (also, there is surgical clips below the coupler). These angiogram show the couplers were effective and operational through the procedure.

In the angiogram analysis, X rays are generated from a Philips apparatus onto the subject (pig). Contrast agent is injected into the subject’s aorta, which shows up on X rays. A series of X rays are taken to generate the angiogram images show in FIGS. 30A and 30B. The images show flow through the vessel, or leak from the vessel and allow assessment of flow through the coupler device.

The many features and advantages of the disclosure are apparent from the detailed specification, and thus, it is intended by the appended claims to cover all such features and advantages of the invention which fall within the true spirit and scope of the disclosure. Further, since numerous modifications and variations will readily occur to those skilled in the art, it is not desired to limit the invention to the exact construction and operation illustrated and described, and accordingly, all suitable modifications and equivalents may be resorted to, falling within the scope of the disclosure.

What is claimed is:

1. A sutureless coupler, comprising:
a member having a passageway,
wherein at least one end of the member is configured for insertion into a vessel, and wherein upon insertion, the member can engage with the vessel.

2. The coupler of claim 1, wherein the member comprises a tubular member.

3. The coupler of claim 1 wherein the member comprises a plurality of protrusions.

3. (canceled)

4. The coupler of claim 1
wherein the member includes one or more protrusions on a surface of at least one end of the member.

5. The coupler of claim 1
wherein the one or more protrusions are present on an outer surface of the member.

6. The coupler of claim 1
wherein the one or more protrusions are present on an inner surface of the member.

7. The coupler of claim 1
wherein the member includes a plurality of protrusions positioned on each end of the member.

8. The coupler of claim 1
wherein the member comprises protrusions having a substantially triangular or wedge shape.

9. The coupler of claim 1
wherein the member comprises protrusions have a substantially acute triangular shape.

10. The coupler of claim 1
wherein a flexible linkage separates a plurality of protrusions positioned on at least one end of the member.

11. The coupler of claim 1
wherein a rigid linkage separates a plurality of protrusions positioned on at least one end of the member.

12. The coupler of claim 1
wherein the member comprises a plurality of protrusions positioned on each end of the tube and each end plurality are separated by at least 2 cm.

13. The coupler of claim 1 wherein the member comprises a plurality of protrusions positioned on each end of the tube and each end plurality are separated by at least 4, 6, 8 or 10 cm.

14. The coupler of claim 1 wherein the member comprises a plurality of protrusions that extend vertically at an acute angle from the member planar surface.

15. The coupler of claim 1
wherein the member comprises 1) a first plurality of protrusions that extend vertically at an acute angle from the member planar surface and 2) a second plurality of protrusions that extend vertically at an acute angle from the member planar surface.

16-28. (canceled)

29. A coupler comprising:
a) a member having a passageway,
b) a first plurality of protrusions that extend vertically at an acute angle from the tube planar surface; and
c) a second plurality of protrusions that extend vertically at an acute angle from the tube planar surface.

30-40. (canceled)

41. The coupler of claim 29
wherein the plurality of protrusions positioned on each end of the tube are separated by at least 2 cm.

42-66. (canceled)

67-76. (canceled)

77. An anastomotic coupling method, comprising:
grasping, by a clamp, a tube having a passageway formed there through and a plurality of protrusions extending from a peripheral surface of each end of the tube;
inserting a first end of the tube into a first vessel to engage the protrusions formed thereon with inner walls of the first vessel;
inserting a second end of the tube into a second vessel to engage the protrusions formed thereon with inner walls of the second vessel; and
releasing the tube from the clamp to join the first vessel and the second vessel.

79-83. (canceled)

84. A method for treating a subject comprising:
contacting a vessel of the subject with a device or system of claim 1.

85-87.

88. A method of bowel surgery, comprising:
contacting a bowel of the subject with a device or system of claim 1 .

89. (canceled)
